(19) United States
(12) Patent Application Publication
(10) Pub. No.: US 2024/0301378 A1
(43) Pub. Date: Sep. 12, 2024

(54) ESTIMATION OF PHENOTYPES USING LARGE-EFFECT EXPRESSION VARIANTS

(71) Applicant: The Board of Trustees of the Leland Stanford Junior University, Stanford, CA (US)

(72) Inventors: Stephen Montgomery, Stanford, CA (US); Craig Small, Stanford, CA (US)

(21) Appl. No.: 18/668,064

(22) Filed: May 17, 2024

Related U.S. Application Data
(63) Continuation of application No. 17/096,636, filed on Nov. 12, 2020.
(60) Provisional application No. 62/934,892, filed on Nov. 13, 2019.

(51) Int. Cl. C12N 9/22; A61K 38/00; C12N 15/86

(52) U.S. Cl. CPC C12N 9/22; C12N 15/86; A61K 38/00; C12N 2710/10043; C12N 2740/15043; C12N 2750/14143

(57) ABSTRACT

Provided here are, inter alia, methods of estimating a genetic predisposition of an individual subject developing a phenotype by identifying a plurality of different rare genetic variant in a population of subjects and estimating the genetic predisposition of the individual subject developing the phenotype based at least in part on the presence of the plurality of different rare genetic variants within the genome of the individual.

### CROSS-REFERENCES TO RELATED APPLICATIONS

This application is a continuation of U.S. patent application Ser. No. 17/096,636 filed Nov. 12, 2020, which claims priority to U.S. Application No. 62,934,892 filed Nov. 13, 2019, which is incorporated herein by reference in entirety and for all purposes.

### STATEMENT AS TO RIGHTS TO INVENTIONS MADE UNDER FEDERALLY SPONSORED RESEARCH AND DEVELOPMENT

This invention was made with Government support under contract HG008150 awarded by the National Institutes of Health. The Government has certain rights in the invention.

### REFERENCE TO A “SEQUENCE LISTING,” A TABLE, OR A COMPUTER PROGRAM LISTING APPENDIX SUBMITTED AS AN ASCII FILE

Table A written in file “Table_A_Obesity.txt” created Nov. 6, 2020, 1,472,420 bytes, machine format IBM-PC, MS Windows operating system, is incorporated by reference herein in its entirety.

Table B written in file “Table_B_Breast_Cancer.txt” created Nov. 6, 2020, 287,755 bytes, machine format IBM-PC, MS Windows operating system, is incorporated by reference herein in its entirety.

Table C written in file “Table C_Type_2_Diabetes.txt” created Nov. 6, 2020, 1,477,071 bytes, machine format IBM-PC, MS Windows operating system, is incorporated by reference hercin in its entirety.

LENGTHY TABLES
The patent application contains a lengthy table section. A copy of the table is available in electronic form from the USPTO web site (https://seqdata.uspto.gov/?pageRequest=docDetail&DocID=US20240301378A1). An electronic copy of the table will also be available from the USPTO upon request and payment of the fee set forth in 37 CFR 1.19(b)(3).

### BACKGROUND

A major goal of complex disease genetics is predicting an individual's disease risk. Recent efforts have aimed at summarizing genome-wide risk for multiple traits and diseases using polygenic risk scores (PRS) (1-6), which are derived by summing genome-wide common genetic variants associated with a given phenotype. PRS have demonstrated stratification of genetic disease risk, but there remains substantial unexplained variability in these predictions. One potential explanation for this variability is the presence of rare variants with large phenotypic effects that are unaccounted for in PRS models (2).

Despite known contributions of rare genetic variants to complex traits and diseases (7,8), rare variants are difficult to robustly characterize and integrate into PRS predictions due to their abundance, poor interpretability and sample size constraints. To in-part alleviate this challenge, it has previously been shown that individuals with outlier gene expression have an increased burden of rare variants proximal to the outlier gene (9-12), and that this subset of rare variants tend to have larger effects on traits and diseases (13,14). This has highlighted that the presence of molecular outliers is indicative of rare variants with the potential for large phenotypic effects.

Given the large effects of rare variants linked to expression outliers, and that these variants are not currently included in existing PRS, the inventors sought to solve the problem in the art of how this subset of rare variants can aid in explaining instances where an individual's phenotype deviates substantially from their phenotype as predicted by PRS and to use this information to improve phenotype prediction.

### BRIEF SUMMARY

Provided herein are methods of estimating a genetic predisposition of an individual subject developing a phenotype by: (i) identifying a plurality of different rare genetic variants in a population of subjects, wherein: (a) each of the plurality of different rare genetic variants is genetically proximal to an expression outlier; (b) each of the plurality of different rare genetic variants has an allelic frequency of less than 1% of the population of subjects; (c) each of the plurality of different rare genetic variants is associated with a phenotype; and (d) each of the expression outliers has an absolute expression Z score from about 1.75 to about 10 across the population of subjects; and (ii) estimating the genetic predisposition of the individual subject developing the phenotype based at least in part on the presence of the plurality of different rare genetic variants within the genome of the individual. This and other embodiments are described in detail herein.

### BRIEF DESCRIPTION OF THE DRAWINGS

FIGS. 1A-1C Linking rare variants associated with gene expression outliers in GTEx to large-scale genetic cohorts. FIG. 1A. Overview of methodology: rare variants (gnomAD MAF<1%) were identified in expression outlier samples across 48 GTEx tissues, discarding any variants also observed in non-outliers; variants from non-outlier samples were selected per gene, matched on gnomAD MAF (<1%) and CADD score (+/−5), to create a control set; variants were linked to UKBB samples, combined with UKBB samples' BMI PRS, ancestry PCs, age, sex, and phenotype data. FIG. 1B. Rate of recovery of rare GTEx rare variants in UKBB imputed set. FIG. 1C. Example gene locus (FOXO3) containing a genome-wide significant hit for BMI illustrates large GWAS effect size of outlier-associated variants: (left) showing distribution of -log10(P-values) for UKBB BMI GWAS for all outlier (gray halo) and non-outlier (striped halo) variants linked to the gene; (right) associated effect sizes, stratified by UKBB allele count. Outlier variants have among the largest effects in gene locus but do not reach genome-wide significance. Points are shaded by LD (1000 Genomes phase 3, European samples) relative to lead variant (top P-value) (triangle) in gene locus.

FIG. 2 Distribution of gnomAD minor allele frequency for outlier and non-outlier variants identified using the top-outlier method.

FIG. 3 Variant count for “top-outlier” and “all-outliers” methods, and sample sizes of UK Biobank and TOPMed WHI cohorts used in the study.

FIGS. 4A-4G Characterizing outlier and non-outlier variants using large-scale GWAS. FIG. 4A. Distribution of odds ratios from permutation testing (N permutations=10,000) to assess relative effect size comparing outlier-and non-outlier variants per gene in UKBB GWAS for BMI (top) and obesity (bottom). Across each permutation, the absolute effect size for a randomly-chosen outlier sample and matched non-outlier sample was obtained for each gene and summed in a contingency matrix to quantify the number of genes where the outlier variant had an absolute effect size greater than the non-outlier variant. This process was repeated for randomly selected non-outlier variants only. P-values were obtained using a Wilcox rank sum test. Subset to genes linked to PRS variants. FIG. 4B. Distribution of odds ratios from permutation testing (N permutations=10,000) (permutation testing method as detailed in (A.)), across progressively more-stringent GTEx outlier Z-scores. Odds ratio increases as a function of outlier Z-score. FIG. 4C. Dispersion of mean effect sizes per gene for outlier and non-outlier variants across genes with variants overlapping a publicly available PRS for BMI, stratified by GTEx outlier Z-score. P-values were obtained using an Ansari Test. FIG. 4D. Distribution of odds ratios from permutation testing (N permutations=10,000) (permutation testing method as detailed in (A.)), using the Million Veteran Project (MVP) GWAS for BMI. FIG. 4E. Distribution of gnomAD allele frequency for outlier-associated (medium gray) and non-outlier (light gray) variants on left side of graph, and variants included in a publicly available PRS for body mass index (dark gray) on right side of graph. Outlier-and non-outlier-associated variants are rarer than variants included in the PRS. FIG. 4F. Example gene locus (FOXO3) containing a genome-wide significant hit in UKBB BMI GWAS (GWAS ID 21001) (triangle). Effect sizes for each variant in locus are displayed on the y-axis and UKBB allele count for each variant is displayed on x-axis.

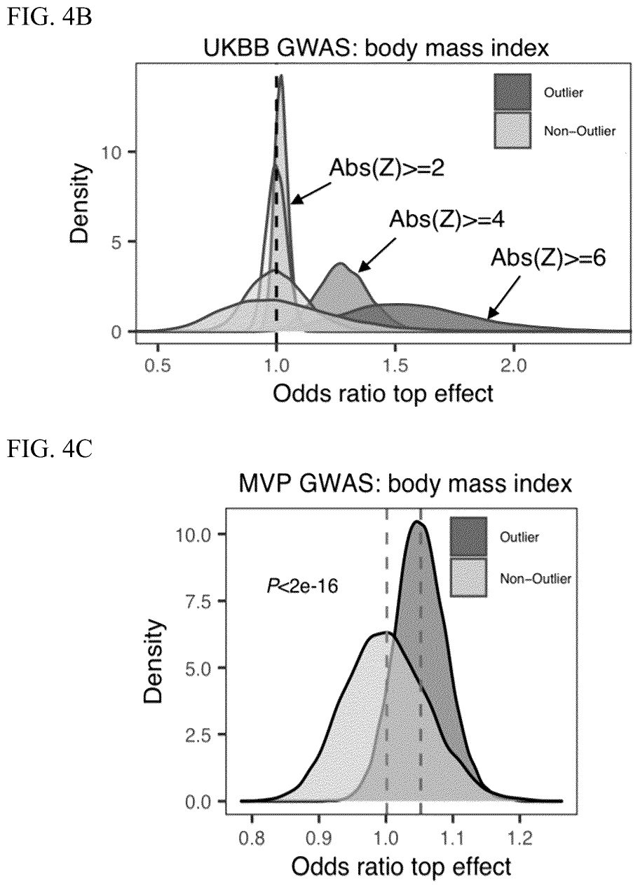

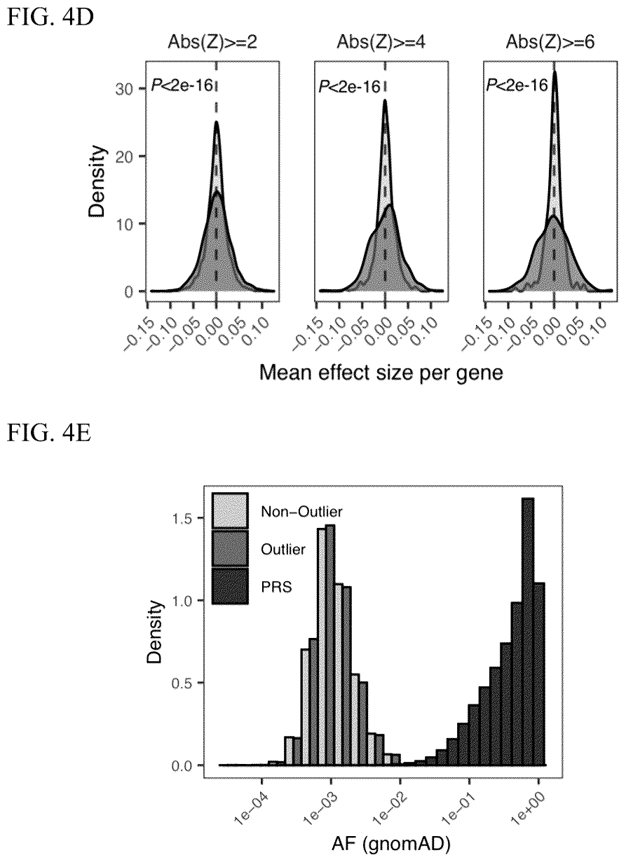

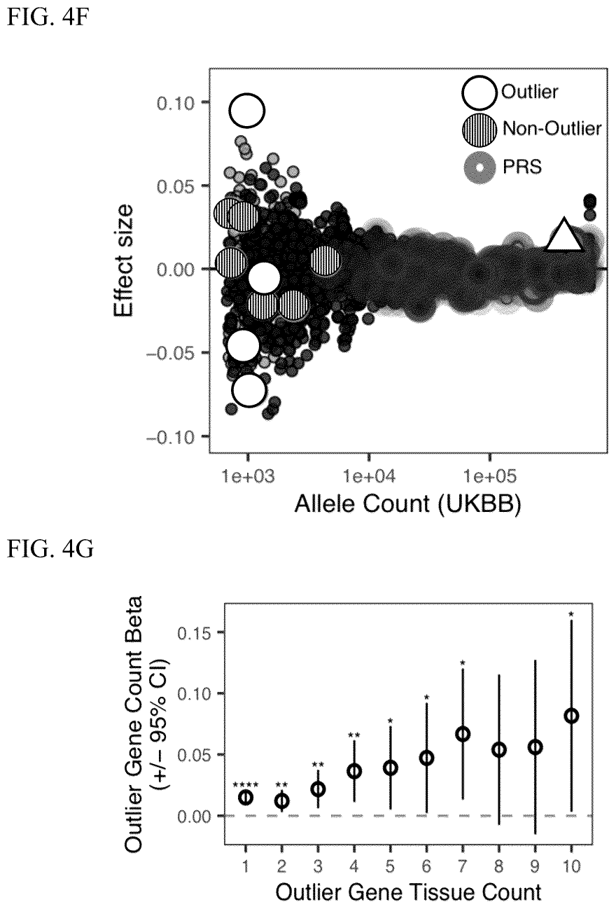

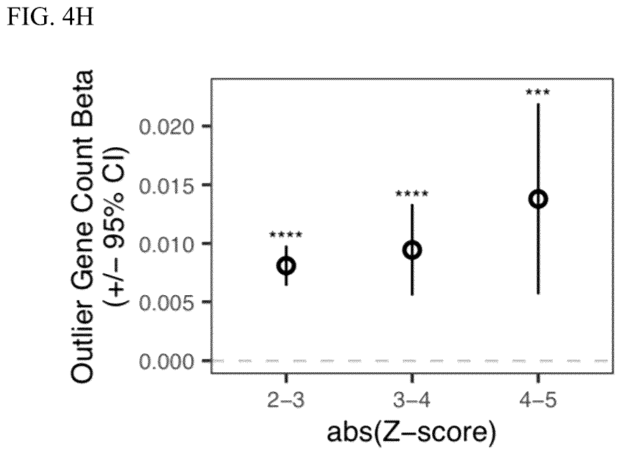

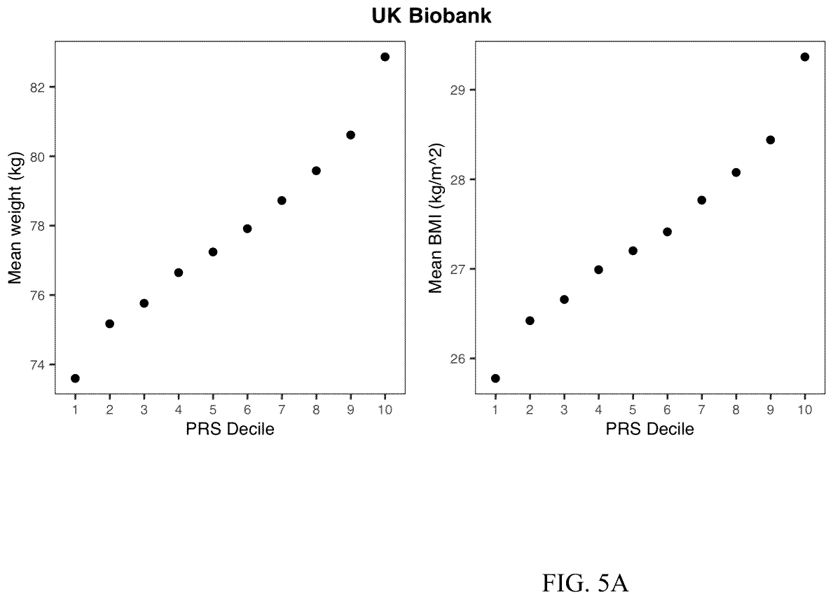

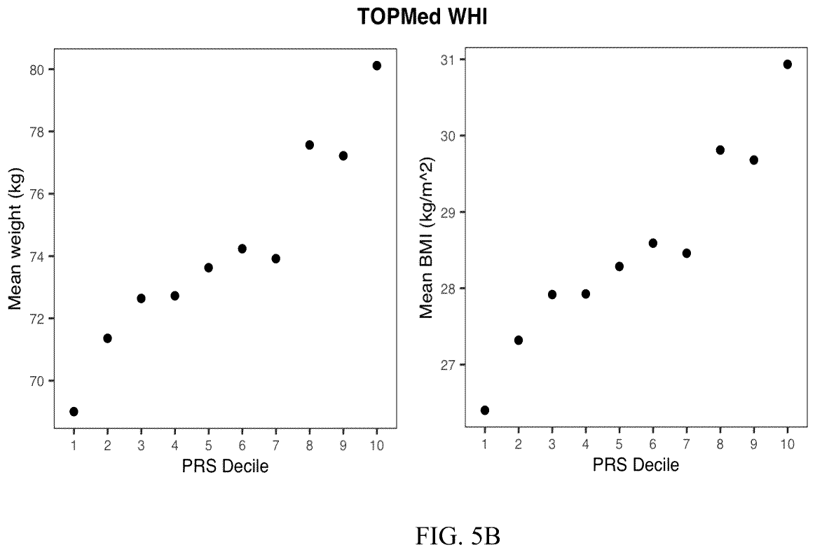

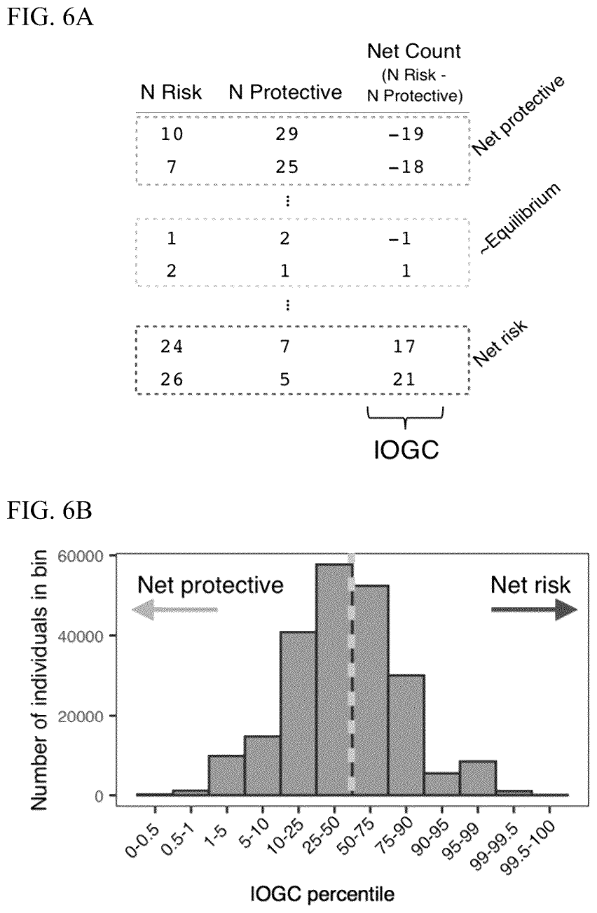

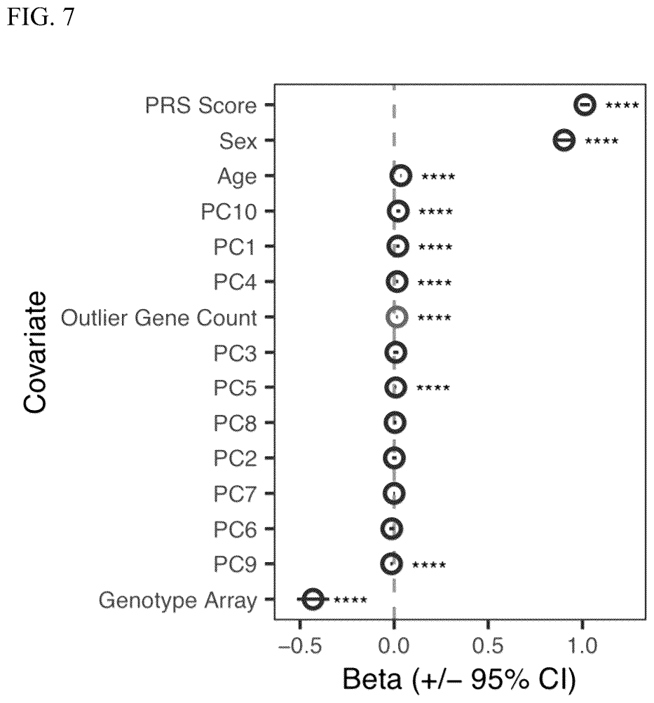

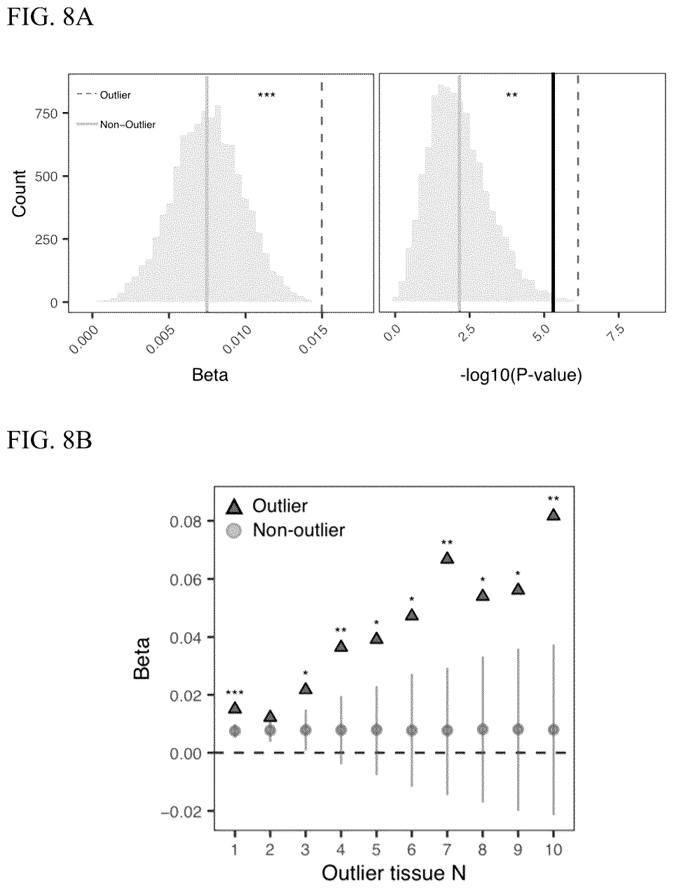

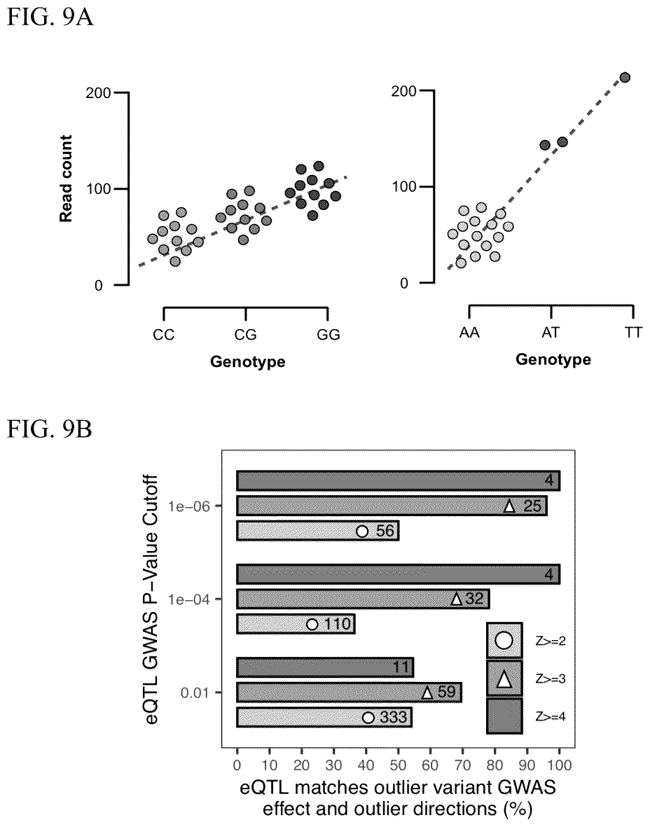

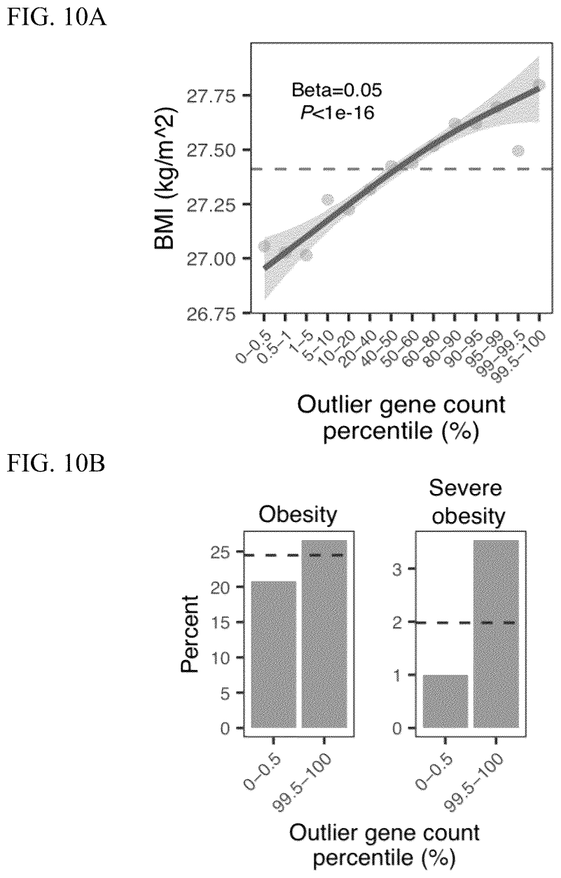

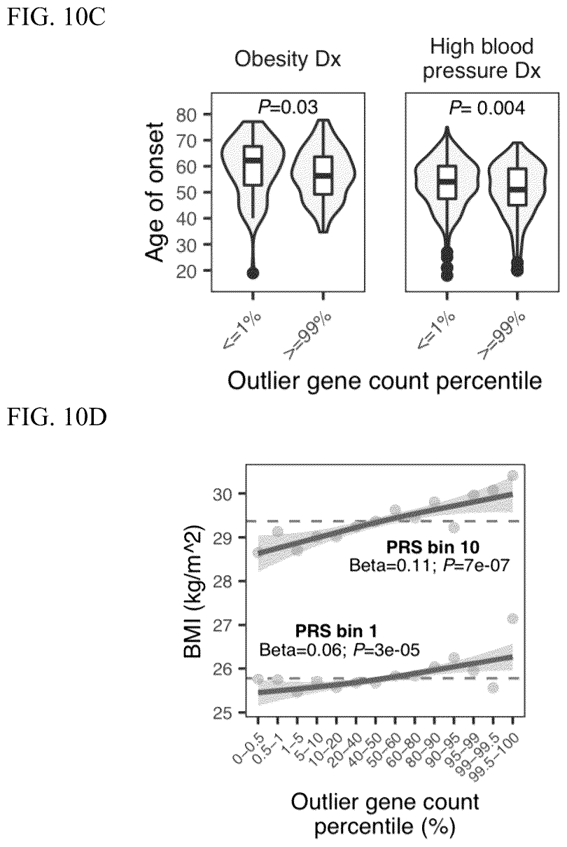

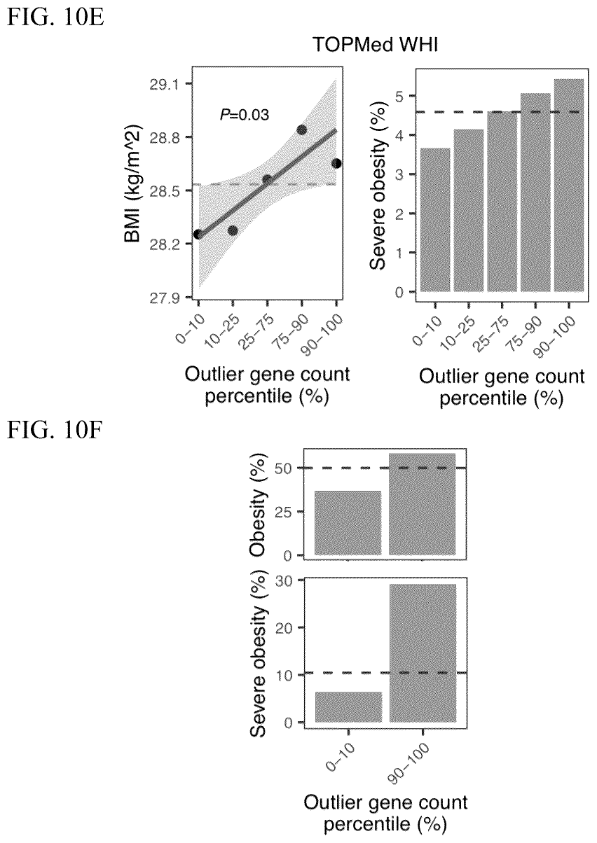

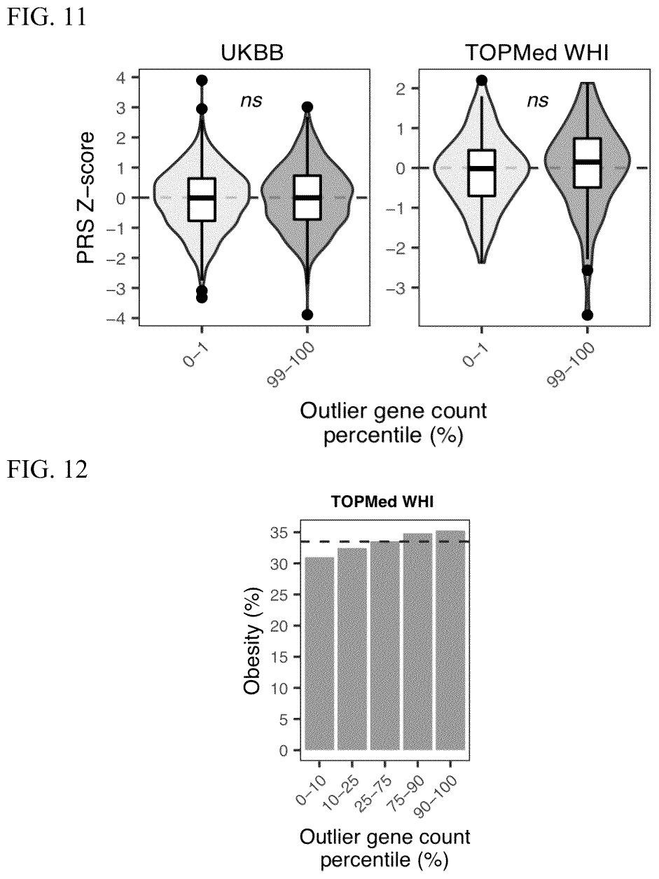

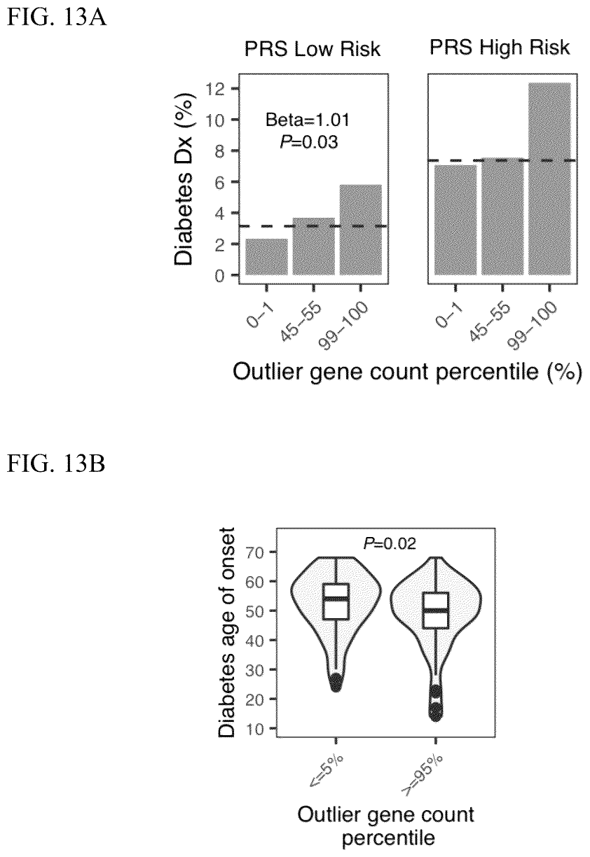

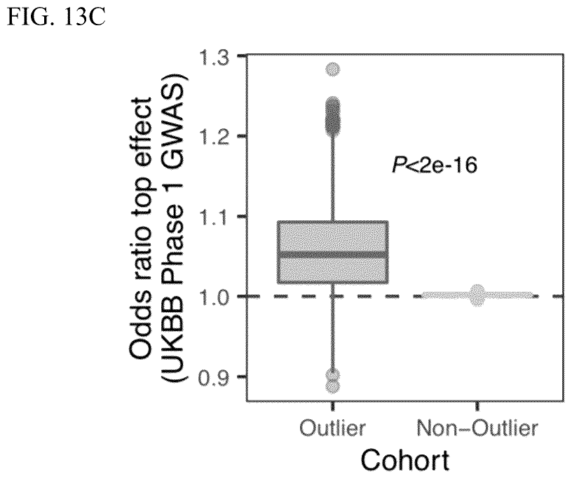

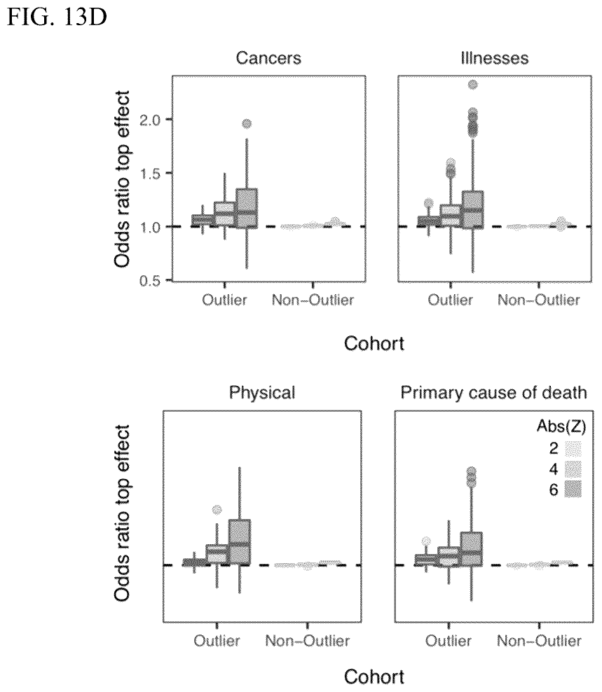

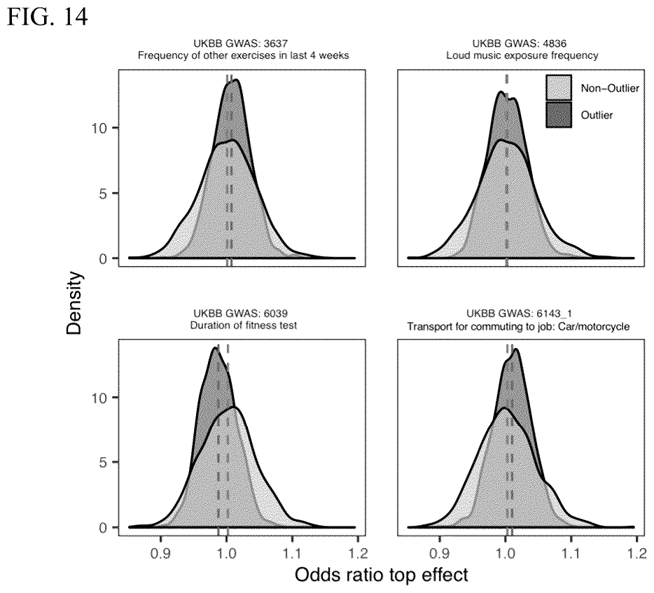

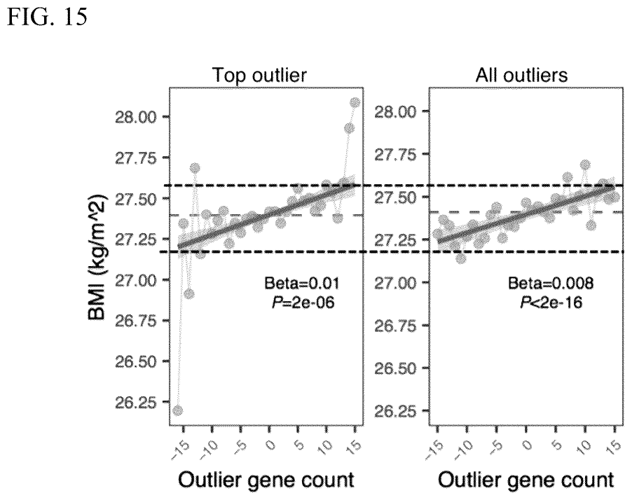

Points are shaded by LD (1000 Genomes phase 3, European). Outlier-associated variants are highlighted in white, non-outlier-associated variants are striped, PRS variants are highlighted in gray. Outlier-associated variants have largest effect sizes in locus. PRS variants tend to be common with small effect size. FIG. 4G. Coefficient estimate for IOGC score increases when subsetting to outlier-associated variants where variants are identified in outliers in an increasing number of GTEx tissues (multi-tissue outlier). FIG. 4H. Mean change in BMI per unit change in IOGC score at difference Z-score cutoffs. Variants identified in more-severe (by Z-score) outliers have larger effects on BMI.

FIGS. 5A-5B Mean weight and BMI for individuals across deciles of a publicly-available polygenic risk score for BMI. FIG. 5A. Mean weight and BMI for individuals in UKBB validation cohort and FIG. 5B. Mean weight and BMI for individuals TOPMed in WHI cohort,

FIGS. 6A-6B Calculating outlier rare variant burden. FIG. 6A. Independent outlier gene count (IOGC) is defined as total outlier variants with a GWAS protective effect subtracted from total outlier variants with a GWAS risk effect, where outlier variants are collapsed to gene-level for individuals with >1 outlier variant per gene; FIG. 6B. Number of individuals in UKBB across different percentiles of IOGC-individuals in bins to left of dotted line have net-protective IOGC scores, whereas individuals in bins to the right have net-risk IOGC score.

FIG. 7 Beta coefficients from a linear regression model testing the effect of outlier gene burden (IOGC score) on body mass index, controlling for the effects of PRS score, sex, age, genotype array, and the first 10 principal components of ancestry.

FIG. 8A. Permutation test (N permutations=10,000) comparing beta coefficients (left) and -log 10(P-value) (right) for IOGC for outlier variants (“top-outlier” method, N variants=8,272) (dashed line) and random samples of non-outlier variants (gray shading; gray line indicates mean across permutations). Coefficients were estimated in a linear regression model controlling for the effects of PRS score, sex, age, genotype array, and first 10 principal components of genetic ancestry. P-value is the empirical P-value. Black line indicates the Bonferroni significance threshold.

FIG. 8B. Permutation test as in (FIG. 8A) as a function of multi-tissue outlier count. Coefficients for outlier variants are indicated in triangles. Coefficients for non-outlier variants are summarized across permutations as mean (+/−SD). Non-outlier variants were randomly sampled to match the total number of outlier variants at each outlier tissue count threshold. P-values are empirical P-values.

FIG. 9A. Leveraging GTEx (v7) eQTL summary statistics to assess concordance in eQTL slope and outlier variant direction (i.e., under-expression or over-expression outlier) and GWAS effect direction.

FIG. 9B. Rate of concordance increases with cQTL GWAS P-value cutoff and outlier variant Z-score threshold. Numbers indicate the total number of variants at the indicated thresholds.

FIGS. 10A-10F Increasing burden of outlier variants is associated with significant deviation in PRS-predicted body mass index. FIG. 10A. Mean BMI at different percentiles of IOGC score and linear regression fit. FIG. 10B. Rate of obesity and severe obesity for individuals with extreme IOGC scores (0.5% and 99.5% percentiles). Logistic regression results: obesity: 1.003 (P=1.20e-14); severe obesity: 1.004 (P=0.0009). FIG. 10C. Age of onset of obesity and high blood pressure diagnosis for individuals with extreme IOGC scores. Obesity dx: percentile <=1%: mean age of onset obesity=59.41; percentile >=99%: mean age of onset 56.95 (P=0.03), mean difference of 2.46 years; high blood pressure dx: percentile <=1%: mean age of onset high blood pressure=53.04; percentile >=99%: 50.44 (P=0.004), mean difference of 2.6 years. FIG. 10D. Mean BMI at different percentiles of IOGC score, computed separately in PRS bins 1 and 10. This demonstrates that the effect of outlier-associated variants is independent from PRS. FIG. 10E. Mean BMI (left) and incidence of severe obesity (right) at different percentiles of IOGC score, including linear regression fit, in TOPMed

WHI; comparing 0-10 and 90-100 percentile bins, there is a 48% higher rate of severe obesity. FIG. 10F. Mean incidence of obesity (top) and severe obesity (bottom) for TOPMed WHI PRS bin 10 cohort, using outlier-associated variants with multi-tissue outlier count>=10.

FIG. 11 Polygenic risk scores for UKBB (left) and TOPMed WHI (right) individuals in top percentiles of IOGC score. No significant differences were observed in either cohort.

FIG. 12 Rates of obesity (BMI>=30 kg/m²) across TOPMed WHI individuals binned by percentiles of IOGC. Black dashed line indicates the mean rate of obesity in the cohort overall.

FIGS. 13A-13D Extending the method to diverse traits and diseases. FIG. 13A. Deviation from PRS-predicted mean incidence of diabetes (%) amongst individuals with extreme IOGC scores, and average score. Logistic regression: beta=1.01 (P=0.03). Dashed line shows the average incidence of diabetes for each PRS bin. Low-risk=PRS bin 1 of 5 (PRS Z-score<−0.84); High-risk=PRS bin 5 of 5 (PRS Z-score>0.84). FIG. 13B. Age on onset of diabetes in PRS z-score>1 cohort: difference in mean age of onset=4.04 years (Wilcox test, P=0.02). FIG. 13C. Distribution of mean odds ratio per UKBB GWAS phenotype across 1,000 permutations for outlier-vs. non-outlier associated variants (left) and non-outlier vs. non-outlier variants (right) (Wilcox test, P<2e-16). FIG. 13D. Distribution of mean odds ratio per UKBB GWAS phenotype (meta-groups: cancer; illnesses; physical; primary cause of death) across 1,000 permutations for outlier-vs. non-outlier associated variants (left side of each graph) and non-outlier vs. non-outlier associated variants. Analysis was repeated at increasing thresholds of outlier gene expression absolute Z-score (from abs(Z-score)>=2 to abs(Z-score)>=6).

FIG. 14 Distribution of odds ratios from permutation testing (N permutations=1,000) comparing GWAS absolute effect sizes of outlier vs. non-outlier variants (dark gray) and non-outlier variants (light gray) across GWAS not expected to be sensitive to gene outlier effects. Both distributions center around an odds ratio=1, indicating no difference in GWAS absolute effect sizes of outlier and non-outlier variants.

FIG. 15 Change in BMI per unit change in IOGC using outlier variants identified using top-outlier (left) and all-outliers (right) methods. IOGC was subset to a fixed range (range=−15:15) to aid comparison. Beta coefficients for IOGC are similar in both methods (top-outlier: linear regression r=0.01; P=2e-06; all-outlier: r=0.008, P<2e=16). Coefficients were estimated in a linear regression model controlling for the effects of PRS score, sex, age, genotype array, and the first 10 principal components of ancestry. Solid lines indicate linear regression fit.

### DETAILED DESCRIPTION

### Definitions

“Table A” refers to the rare genetic variants identified by the inventors for obesity.

“Table B” refers to the rare genetic variants identified by the inventors for breast cancer.

“Table C” refers to the rare genetic variants identified by the inventors for type 2 diabetes.

The term “subject” refers to a living organism suffering from or prone to a disease or condition that can be monitored and/or treated by administration of behavioral modifications, prophylactic therapy, pharmaceutical compositions, and the like. Non-limiting examples of a “subject” include humans, other mammals, bovines, rats, mice, dogs, monkeys, goat, sheep, cows, deer, and other non-mammalian animals. In embodiments, the subject is human.

The term “population of subjects” refers to more than one subject. In embodiments, “population of subjects” refers to more than 50 subjects, more than 100 subjects, more than 500 subjects, more than 1,000 subjects, more than 10,000 subjects, more than 100,000 subjects, more than 500,000 subjects, or more than 1 million subjects. In embodiments, more than 50% of the “population of subjects” have the same gender, nationality, race, or a combination thereof. In embodiments, more than 75% of the “population of subjects” have the same gender, nationality, race, or a combination thereof. In embodiments, more than 90% of the “population of subjects” have the same gender, nationality, race, or a combination thereof. In embodiments, the population of subjects refers to the subjects in a genome wide association study database. In embodiments, the population of subjects refers to the subjects in a biobank. In embodiments, the population of subjects refers to the subjects in the UK Biobank, Million Veterans Project, TOPMed Women's Health Initiative, or a combination thereof. In embodiments, the population of subjects refers to a sub-group of subjects in the UK Biobank, Million Veterans Project, TOPMed Women's Health Initiative, or a combination thereof. In embodiments, the “population of subjects” is referred to as a cohort.

The term “individual subject” refers to one (1) subject. In embodiments, an “individual subject” and more than 50% of the “population of subjects” have the same gender, race, nationality, or a combination of two or more thereof. In embodiments, an “individual subject” and more than 75% of the “population of subjects” have the same gender, race, nationality, or a combination of two or more thereof. In embodiments, an “individual subject” and more than 90% of the “population of subjects” have the same gender, race, nationality, or a combination of two or more thereof. In embodiments, an “individual subject” and the “population of subjects” have the same gender, race, nationality, or a combination of two or more thereof.

The term “genetic predisposition” refers to the likelihood or chance of a subject developing a particular phenotype based on the subject's genetic makeup. Genetic predisposition is based, at least in part, on the subject's rare genetic variants.

The “genome” of an individual subject can comprise that individual subject's complete set of chromosomes, including both coding and non-coding regions. Particular locations within the genome of a species are referred to as “loci,” “sites,” or “features.”

The term “gene” refers to a DNA sequence in a chromosome that codes for a product (either RNA or its translation product, a polypeptide). A gene contains a coding region and includes regions preceding and following the coding region. The coding region is comprised of a plurality of coding segments (“exons”) and intervening sequences (“introns”) between individual coding segments. A gene can also comprise a non-coding RNA product such as a miRNA or IncRNA gene.

The term “chromosome” as used herein refers to a gene carrier of a cell that is derived from chromatin and comprises DNA and protein components (e.g., histones). The conventional internationally recognized individual human genome chromosome numbering identification system is employed herein. The size of an individual chromosome can vary from one type to another with a given multi- chromosomal genome and from one genome to another. In the case of the human genome, the entire DNA mass of a given chromosome is usually greater than about 100,000,000 base pairs.

The term “allele” refers to varying forms of the genomic DNA located at a given site. In embodiments, “allele” refers to one of a pair or series of genetic variants of a polymorphism at a specific genomic location. In the case of a site where there are two distinct alleles in a species, referred to as “A” and “B”, each individual member of the species can have one of four possible combinations: AA; AB; BA; and BB. The first allele of each pair is inherited from one parent, and the second from the other. The term “allelic frequency” refers to the incidence of a gene variant in a population of subjects. Alleles are variant forms of a gene that are located at the same position, or genetic locus, on a chromosome. An allele frequency is calculated by dividing the number of times the allele of interest is observed in a population of subjects by the total number of copies of all the alleles at that particular genetic locus in the population of subjects.

“Genotype” refers to the chemical composition of polynucleotide sequences within the genome of an individual. In embodiments, the genotype comprises SNVs, single nucleotide polymorphisms (SNPs), indels, and/or CNVs.

As used herein, a “haplotype” is one or a set of signature genetic changes (polymorphisms) that are normally grouped closely together on the DNA strand, and are usually inherited as a group; the polymorphisms are also referred to herein as “markers.” A “haplotype” as used herein is information regarding the presence or absence of one or more genetic markers in a given chromosomal region in a subject. A haplotype can consist of a variety of genetic markers, including indels (insertions or deletions of the DNA at particular locations on the chromosome); single nucleotide polymorphisms (SNPs) in which a particular nucleotide is changed; microsatellites; and minis satellites.

The term “phenotype” or “phenotypic trait” refers to one or more characteristics of a subject. A phenotype of a subject can be the composite of the subject's observable characteristics, which may result from the expression of the subject's genes and, in some cases, the influence of environmental factors and the interactions between the two. A subject's phenotype can be driven by constituent proteins in the subject's “proteome,” which is the collection of all proteins produced by the cells comprising the subject and coded for in the subject's genome. The proteome can also be defined as the collection of all proteins expressed in a given cell type within a subject. A disease can be a phenotype. In embodiments, the phenotype refers to a disease that a subject has or that a subject is predisposed to having. In embodiments, the phenotype refers a subject's responsiveness to treatment with a certain drug, i.e., that the subject is more or less likely to benefit from being administered a certain drug.

The term “expression outlier” refers to genes or gene-related molecular measurements (such as alternative-splicing, methylation, chromatin structure, chromatin accessibility, allele-specific expression, protein-levels) that have increased expression or decreased expression in a subset of samples (e.g., individual subjects having a certain phenotype) compared to a control (e.g., a population of subjects). In embodiments, an “expression outlier” has an absolute expression Z score from 1.75 to 10 across a population of subjects. In embodiments, the gene-related molecular measurements are referred to as a signature or a gene signature.

The terms “increased expression” and “decreased expression” refers to an expression level of a biomarker in the subject's sample as compared to a control level representing the same biomarker. In embodiments, the control level is a level of expression from population of subjects. The term “absolute expression Z score” refers to a measurement of an expression outlier for a given individual, compared with the remainder of samples in a cohort.

The term “chromatin structure” refers to the arrangement of the complex comprising DNA and proteins (i.e., histones) that form chromosomes within cukaryotic cells. Chromatin structure is dynamic and involves processes such as chromatin remodeling. Variations in chromatin structure, for example transitions to cuchromatin or heterochromatin, are associated with functions including DNA replication and gene expression, where the functions are correlated with the ability of various transcription factors and other proteins to access chromatinized DNA. Modifications that result in chromatin structural changes include histone acetylation, methylation, phosphorylation, ubiquitination, SUMOylation, ADP ribosylation, deamination, and proline isomerization.

The term “chromatin accessibility” refers to the degree to which nuclear macromolecules are able to contact DNA in the chromatin complex. Accessible chromatin allows for interaction of transcription factors and recruitment of other macromolecules, which regulate processes including gene expression.

The term “DNA methylation” or “methylation” refers to refers to the process wherein methyl groups are added to a DNA molecule. In humans, DNA methylation most commonly occurs at the C5 position of cytosine in CpG sites. DNA methylation in gene promoter regions is typically associated with decreased gene expression, whereas in exons and introns of a gene, DNA methylation is associated with increased transcription of the gene.

The term “RNA splicing” or “splicing” refers to RNA processing, wherein a precursor mRNA is made into a mature mRNA. During splicing, non-coding regions of RNA (introns) are removed and coding regions (exons) are connected. Splicing is catalyzed by the spliceosome complex and typically occurs during transcription of the precursor mRNA.

A “variant” or “genetic variant” refers to any change in an individual nucleotide sequence compared to a control sequence. In embodiments, the term “variant” and “genetic variant” refer to a nucleic acid molecule comprising a polymorphism. A variant can be a structural variant or copy number variant, which can be genomic variants that are larger than single nucleotide variants or short indels. A variant can be an alteration or polymorphism in a nucleic acid sample or genome of a subject. Single nucleotide polymorphisms (SNPs) are a form of polymorphisms. Polymorphisms can include single nucleotide variations (SNVs), multi-nucleotide variants (MVSs), insertions, deletions, repeats, small insertions, small deletions, small repeats, structural variant junctions, variable length tandem repeats, and/or flanking sequences. Copy number variants (CNVs), transversions and other rearrangements are also forms of genetic variation. A genomic alternation may be a base change, insertion, deletion, repeat, copy number variation, or transversion.

A “single nucleotide polymorphism,” “SNP,” “single nucleotide variant” or “SNV” refer to a single nucleotide within the individual sequence that is changed in comparison to the reference sequence. SNPs that occur in the protein coding regions of genes that give rise to the expression of variant or defective proteins are potentially the cause of a genetic-based disease. Even SNPs that occur in non-coding regions can result in altered mRNA and/or protein expression. Examples are SNPs that defective splicing at exon/intron junctions. Exons are the regions in genes that contain three-nucleotide codons that are ultimately translated into the amino acids that form proteins. Introns are regions in genes that can be transcribed into pre-messenger RNA but do not code for amino acids. In the process by which genomic DNA is transcribed into messenger RNA, introns are often spliced out of pre-messenger RNA transcripts to yield messenger RNA. A SNP can be in a coding region or a non-coding region. A SNP in a coding region can be a silent mutation, otherwise known as a synonymous mutation, wherein an encoded amino acid is not changed due to the variant. A SNP in a coding region can be a missense mutation, wherein an encoded amino acid is changed due to the variant. A SNP in a coding region can also be a nonsense mutation, wherein the variant introduces a premature stop codon.

“Indel” refers to an insertion or a deletion of a nucleobase within a polynucleotide sequence. An indel can be a frame-shift mutation or a splice-site mutation. In embodiments, an indel is an insertion of a nucleobase within a polynucleotide sequence. In embodiments, an indel is a deletion of a nucleobase within a polynucleotide sequence.

“Copy number variant” or “CNV” refers a phenomenon in which sections of a polynucleotide sequence are repeated or deleted, the number of repeats in the genome varying between individuals in a population of subjects. In embodiments, the section of the polynucleotide sequence is short, comprising about two nucleotides (bi-nucleotide CNV) or three nucleotides (tri-nucleotide CNV). In embodiments, the section of the polynucleotide sequence is long, comprising a number of nucleotides between four nucleotides and an entire length of a genc.

The term “common genetic variant” refers to a genetic variant that has an allelic frequency of 1% or more in a population of subjects. In embodiments, a “common genetic variant” refers to a genetic variant that has an allelic frequency of 5% or more in a population of subjects. In embodiments, a “common genetic variant” refers to a genetic variant that has an allelic frequency of 5% or more in a population of subjects and is associated with a phenotype. In embodiments, a “common genetic variant” refers to a genetic variant that has an allelic frequency of 5% or more in a population of subjects and is associated with a phenotype.

The term “rare genetic variant” refers to a genetic variant that has an allelic frequency of less than 1% in a population of subjects. In embodiments, “rare genetic variant” refers to a genetic variant that is proximal to an expression outlier and that has an allelic frequency of less than 1% in a population of subjects. In embodiments, “rare genetic variant” refers to a genetic variant that is proximal to an expression outlier, that has an allelic frequency of less than 1% in a population of subjects, and is associated with a phenotype.

The term “genetically proximal” refers to the distance between a rare genetic variant and an expression outlier. In embodiments, the term “genetically proximal” refers to a rare genetic variant that is within 300 kilobases of an expression outlier.

The term “associated” or “associated with” means that a phenotype (e.g., disease, such as obesity, cancer, type 2 diabetes) is caused by (in whole or in part), or a symptom of the disease is caused by (in whole or in part), a rare genetic variant.

### Methods

Provided herein are methods and systems of estimating a genetic predisposition of an individual subject developing a phenotype or calculating a risk score representing the likelihood that an individual subject will develop a specific phenotypic trait. The risk score is based one or more rare genetic variants present in the genome of the individual subject. In embodiments, the one or more rare genetic variants is detected in a biological sample obtained from the individual subject. In embodiments, the one or more rare genetic variants comprise a SNV, an indel, and/or a CNV. In embodiments, the one or more rare genetic variants present in the genotype of the individual are associated with an increased likelihood that the individual has, or will develop, a specific phenotypic trait. In embodiments, the one or more rare genetic variants present in the genotype of the individual are associated with a decreased likelihood that the individual has, or will develop, a specific phenotypic trait. In embodiments, the phenotypic trait comprises a clinical trait (e.g., disease, responsiveness to medications, allergies to medications), a physical exercise trait, a skin trait, a hair trait, an allergy trait, a nutrition trait (e.g., food allergy, or a mental trait).

In embodiments, the disclosure provides method of estimating a genetic predisposition of an individual subject developing a phenotype by identifying a plurality of different rare genetic variant in a population of subjects, and estimating the genetic predisposition of the individual subject developing the phenotype based at least in part on the presence of the plurality of different rare genetic variants within the genome of the individual. In embodiments, each of the plurality of different rare genetic variants is genetically proximal to an expression outlier. In embodiments, each of the plurality of different rare genetic variants has an allelic frequency of less than 1% of the population of subjects. In embodiments, each of the plurality of different rare genetic variants is associated with a phenotype. In embodiments, each of the expression outliers has an absolute expression Z score between 1.75 and 10 across the population of subjects.

In embodiments, the disclosure provides method of estimating a genetic predisposition of an individual subject developing a phenotype by identifying a plurality of different rare genetic variant in a population of subjects, identifying a plurality of different common genetic variants in a population of subjects, and estimating the genetic predisposition of the individual subject developing the phenotype based at least in part on the presence of the plurality of different rare genetic variants within the genome of the individual. In embodiments, each of the plurality of different rare genetic variants is genetically proximal to an expression outlier. In embodiments, each of the plurality of different rare genetic variants has an allelic frequency of less than 1% of the population of subjects. In embodiments, each of the plurality of different rare genetic variants is associated with a phenotype. In embodiments, each of the expression outliers has an absolute expression Z score between 1.75 and 10 across the population of subjects. In embodiments, the plurality of different common genetic variants has an allelic frequency greater than 1% of the population of subjects. the plurality of different common genetic variants has an allelic frequency greater than 5% of the population of subjects.

In embodiments, the disclosure provides method of estimating a genetic predisposition of an individual subject developing a phenotype by: (i) identifying a plurality of different rare genetic variant in a population of subjects, wherein: (a) each of the plurality of different rare genetic variants is genetically proximal to an expression outlier; (b) each of the plurality of different rare genetic variants has an allelic frequency of less than 1% of the population of subjects; (c) each of the plurality of different rare genetic variants is associated with a phenotype; and (d) each of the expression outliers has an absolute expression Z score between 1.75 and 10 across the population of subjects; and (ii) estimating the genetic predisposition of the individual subject developing the phenotype based at least in part on the presence of the plurality of different rare genetic variants within the genome of the individual.

In embodiments, the disclosure provides method of estimating a genetic predisposition of an individual subject developing a phenotype by: (i) identifying a plurality of different rare genetic variant in a population of subjects, wherein: (a) each of the plurality of different rare genetic variants is genetically proximal to an expression outlier; (b) each of the plurality of different rare genetic variants has an allelic frequency of less than 1% of the population of subjects; (c) each of the plurality of different rare genetic variants is associated with a phenotype; and (d) each of the expression outliers has an absolute expression Z score between 1.75 and 10 across the population of subjects; (ii) identifying a plurality of different common genetic variants in a population of subjects, wherein each of the plurality of different common genetic variants has an allelic frequency greater than 1% of the population of subjects; and (iii) estimating the genetic predisposition of the individual subject developing the phenotype based on the presence of the plurality of common genetic variants within the genome of the individual and the plurality of different rare genetic variants within the genome of the individual.

In embodiments of the methods described herein, the plurality of different rare genetic variants is genetically proximal to an expression outlier. In embodiments, the plurality of different rare genetic variants are within 300 kilobases of the expression outlier. In embodiments, the plurality of different rare genetic variants are within 250 kilobases of the expression outlier. In embodiments, the plurality of different rare genetic variants are within 200 kilobases of the expression outlier. In embodiments, the plurality of different rare genetic variants are within 190 kilobases of the expression outlier. In embodiments, the plurality of different rare genetic variants are within 180 kilobases of the expression outlier. In embodiments, the plurality of different rare genetic variants are within 170 kilobases of the expression outlier. In embodiments, the plurality of different rare genetic variants are within 160 kilobases of the expression outlier. In embodiments, the plurality of different rare genetic variants are within 150 kilobases of the expression outlier. In embodiments, the plurality of different rare genetic variants are within 140 kilobases of the expression outlier. In embodiments, the plurality of different rare genetic variants are within 130 kilobases of the expression outlier. In embodiments, the plurality of different rare genetic variants are within 120 kilobases of the expression outlier. In embodiments, the plurality of different rare genetic variants are within 110 kilobases of the expression outlier. In embodiments, the plurality of different rare genetic variants are within 100 kilobases of the expression outlier. In embodiments, the plurality of different rare genetic variants are within 95 kilobases of the expression outlier. In embodiments, the plurality of different rare genetic variants are within 90 kilobases of the expression outlier. In embodiments, the plurality of different rare genetic variants are within 85 kilobases of the expression outlier. In embodiments, the plurality of different rare genetic variants are within 80 kilobases of the expression outlier. In embodiments, the plurality of different rare genetic variants are within 75 kilobases of the expression outlier. In embodiments, the plurality of different rare genetic variants are within 70 kilobases of the expression outlier. In embodiments, the plurality of different rare genetic variants are within 65 kilobases of the expression outlier. In embodiments, the plurality of different rare genetic variants are within 60 kilobases of the expression outlier. In embodiments, the plurality of different rare genetic variants are within 55 kilobases of the expression outlier. In embodiments, the plurality of different rare genetic variants are within 50 kilobases of the expression outlier. In embodiments, the plurality of different rare genetic variants are within 45 kilobases of the expression outlier. In embodiments, the plurality of different rare genetic variants are within 40 kilobases of the expression outlier. In embodiments, the plurality of different rare genetic variants are within 35 kilobases of the expression outlier. In embodiments, the plurality of different rare genetic variants are within 30 kilobases of the expression outlier. In embodiments, the plurality of different rare genetic variants are within 25 kilobases of the expression outlier. In embodiments, the plurality of different rare genetic variants are within 20 kilobases of the expression outlier. In embodiments, the plurality of different rare genetic variants are within 15 kilobases of the expression outlier. In embodiments, the plurality of different rare genetic variants are within 10 kilobases of the expression outlier. In embodiments, the plurality of different rare genetic variants are within 5 kilobases of the expression outlier. In embodiments, the plurality of different rare genetic variants are within about 1 kilobase to about 25 kilobases of the expression outlier. In embodiments, the plurality of different rare genetic variants are within about 1 kilobase to about 20 kilobases of the expression outlier. In embodiments, the plurality of different rare genetic variants are within about 1 kilobase to about 15 kilobases of the expression outlier. In embodiments, the plurality of different rare genetic variants are within about 1 kilobase to about 10 kilobases of the expression outlier. In embodiments, the plurality of different rare genetic variants are within about 0.1 kilobases to about 100 kilobases of the expression outlier. In embodiments, the plurality of different rare genetic variants are within about 0.1 kilobases to about 50 kilobases of the expression outlier. In embodiments, the plurality of different rare genetic variants are within about 0.1 kilobases to about 45 kilobases of the expression outlier. In embodiments, the plurality of different rare genetic variants are within about 0.1 kilobases to about 40 kilobases of the expression outlier. In embodiments, the plurality of different rare genetic variants are within about 0.1 kilobases to about 35 kilobases of the expression outlier. In embodiments, the plurality of different rare genetic variants are within about 0.1 kilobases to about 30 kilobases of the expression outlier. In embodiments, the plurality of different rare genetic variants are within about 0.1 kilobases to about 25 kilobases of the expression outlier. In embodiments, the plurality of different rare genetic variants are within about 0.1 kilobases to about 20 kilobases of the expression outlier. In embodiments, the plurality of different rare genetic variants are within about 1 kilobase to about 15 kilobases of the expression outlier. In embodiments, the plurality of different rare genetic variants are within about 1 kilobase to about 10 kilobases of the expression outlier. In embodiments, the plurality of different rare genetic variants are adjacent to or within about 25 kilobases of the expression outlier. In embodiments, the plurality of different rare genetic variants are adjacent to or within to about 20 kilobases of the expression outlier. In embodiments, the plurality of different rare genetic variants are adjacent to or within about 15 kilobases of the expression outlier. In embodiments, the plurality of different rare genetic variants are adjacent to or within about 10 kilobases of the expression outlier.

In embodiments of the methods described herein, the rare genetic variant is a nucleotide variant, an indel, a copy number variation, a duplication, a translocation, an inversion, or a combination of two or more thereof. In embodiments, the rare genetic variant is a nucleotide variant. In embodiments, the rare genetic variant is an indel. In embodiments, the indel is an insertion. In embodiments, the indel is a deletion. In embodiments, the rare genetic variant is a copy number variation. In embodiments, the rare genetic variant is a duplication. In embodiments, the rare genetic variant is a translocation. In embodiments, the rare genetic variant is an inversion. In embodiments, the nucleotide variant is a single nucleotide variant. In embodiments, the nucleotide variant is a multi-nucleotide variant. In embodiments, the nucleotide variant is a single nucleotide polymorphism.

In embodiments of the methods described herein, the expression outliers have an absolute expression Z score from 1.75 to 10 across the population of subjects. In embodiments, the expression outliers have an absolute expression Z score from 2 to 10 across the population of subjects. In embodiments, the expression outliers have an absolute expression Z score from 3 to 10 across the population of subjects. In embodiments, the expression outliers have an absolute expression Z score from 4 to 10 across the population of subjects. In embodiments, the expression outliers have an absolute expression Z score from 5 to 10 across the population of subjects. In embodiments, the expression outliers have an absolute expression Z score from 2 to 3 across the population of subjects. In embodiments, the expression outliers have an absolute expression Z score from 3 to 4 across the population of subjects. In embodiments, the expression outliers have an absolute expression Z score from 4 to 5 across the population of subjects. In embodiments, the expression outliers have an absolute expression Z score from 2 to 5 across the population of subjects. In embodiments, the expression outliers have an absolute expression Z score from 2 to 4 across the population of subjects. In embodiments, the expression outliers have an absolute expression Z score from 3 to 5 across the population of subjects.

In embodiments of the methods described herein, the expression outlier has a gene-related molecular measurement (such as alternative-splicing, methylation, chromatin accessibility, allele-specific expression, protein levels, RNA levels) that is over-expressed or under-expressed in a subset of samples relative to a population of subjects. In embodiments, the gene-related molecular measurement is alternative-splicing, methylation, chromatin accessibility, allele-specific expression, protein levels, or RNA levels. In embodiments, the gene-related molecular measurement is alternative-splicing. In embodiments, the gene-related molecular measurement is methylation. In embodiments, the gene-related molecular measurement is chromatin accessibility. In embodiments, the gene-related molecular measurement is allele-specific expression. In embodiments, the gene-related molecular measurement is protein levels. In embodiments, the gene-related molecular measurement is RNA levels. In embodiments, the expression outlier has an increased RNA expression level, a decreased RNA expression level, an increased protein expression level, or a decreased protein expression level. In embodiments, the expression outlier has an increased RNA expression level. In embodiments, the expression outlier has a decreased RNA expression level. In embodiments, the expression outlier has an increased protein expression level. In embodiments, the expression outlier has a decreased protein expression level. In embodiments, the signature is compared to a control.

In embodiments of the methods described herein, the individual subject and at least 50% of the population of subjects have the same gender, race, nationality, or a combination of two or more thereof. In embodiments, the individual subject and at least 50% of the population of subjects have the same gender. In embodiments, the individual subject and at least 50% of the population of subjects have the same race. In embodiments, the individual subject and at least 50% of the population of subjects have the same nationality. In embodiments, the individual subject and more than 50% of the population of subjects have the same gender, race, nationality, or a combination of two or more thereof. In embodiments, the individual subject and more than 50% of the population of subjects have the same gender. In embodiments, the individual subject and more than 50% of the population of subjects have the same race. In embodiments, the individual subject and more than 50% of the population of subjects have the same nationality. In embodiments, the individual subject and at least 60% of the population of subjects have the same gender, race, nationality, or a combination of two or more thereof. In embodiments, the individual subject and at least 60% of the population of subjects have the same gender. In embodiments, the individual subject and at least 60% of the population of subjects have the same race. In embodiments, the individual subject and at least 60% of the population of subjects have the same nationality. In embodiments, the individual subject and at least 65% of the population of subjects have the same gender, race, nationality, or a combination of two or more thereof. In embodiments, the individual subject and at least 65% of the population of subjects have the same gender. In embodiments, the individual subject and at least 65% of the population of subjects have the same race. In embodiments, the individual subject and at least 65% of the population of subjects have the same nationality. In embodiments, the individual subject and at least 70% of the population of subjects have the same gender, race, nationality, or a combination of two or more thereof. In embodiments, the individual subject and at least 70% of the population of subjects have the same gender. In embodiments, the individual subject and at least 70% of the population of subjects have the same race. In embodiments, the individual subject and at least 70% of the population of subjects have the same nationality. In embodiments, the individual subject and at least 75% of the population of subjects have the same gender, race, nationality, or a combination of two or more thereof. In embodiments, the individual subject and at least 75% of the population of subjects have the same gender. In embodiments, the individual subject and at least 75% of the population of subjects have the same race. In embodiments, the individual subject and at least 75% of the population of subjects have the same nationality. In embodiments, the individual subject and at least 80% of the population of subjects have the same gender, race, nationality, or a combination of two or more thereof. In embodiments, the individual subject and at least 80% of the population of subjects have the same gender. In embodiments, the individual subject and at least 80% of the population of subjects have the same race. In embodiments, the individual subject and at least 80% of the population of subjects have the same nationality. In embodiments, the individual subject and at least 85% of the population of subjects have the same gender, race, nationality, or a combination of two or more thereof. In embodiments, the individual subject and at least 85% of the population of subjects have the same gender. In embodiments, the individual subject and at least 85% of the population of subjects have the same race. In embodiments, the individual subject and at least 85% of the population of subjects have the same nationality. In embodiments, the individual subject and at least 90% of the population of subjects have the same gender, race, nationality, or a combination of two or more thereof. In embodiments, the individual subject and at least 90% of the population of subjects have the same gender. In embodiments, the individual subject and at least 90% of the population of subjects have the same race. In embodiments, the individual subject and at least 90% of the population of subjects have the same nationality. In embodiments, the individual subject and at least 95% of the population of subjects have the same gender, race, nationality, or a combination of two or more thereof. In embodiments, the individual subject and at least 95% of the population of subjects have the same gender. In embodiments, the individual subject and at least 95% of the population of subjects have the same race. In embodiments, the individual subject and at least 95% of the population of subjects have the same nationality. In embodiments, the individual subject and the population of subjects have the same gender, race, nationality, or a combination of two or more thereof. In embodiments, the individual subject and the population of subjects have the same gender. In embodiments, the individual subject and the population of subjects have the same race. In embodiments, the individual subject and the population of subjects have the same nationality.

In embodiments of the methods described herein, the plurality of different common genetic variants has an allelic frequency of 1% or more of the population of subjects. In embodiments, the plurality of different common genetic variants has an allelic frequency of 2% or more of the population of subjects. In embodiments, the plurality of different common genetic variants has an allelic frequency of 3% or more of the population of subjects. In embodiments, the plurality of different common genetic variants has an allelic frequency of 4% or more of the population of subjects. In embodiments, the plurality of different common genetic variants has an allelic frequency of 5% or more of the population of subjects. In embodiments, the plurality of different common genetic variants has an allelic frequency greater than 5% of the population of subjects.

In embodiments for estimating the genetic predisposition of the individual subject developing the phenotype based on the presence of the plurality of common genetic variants within the genome of the individual and the plurality of different rare genetic variants within the genome of the individual, such an estimate can be conducted using linear regression.

### Computer Systems

In embodiments, the disclosure provides a computer program product comprising machine-readable medium storing instructions that, when executed by at least one programmable processor, cause the at least one programmable processor to perform operations comprising the methods described herein, including all embodiments thereof.

In embodiments, the disclosure provides a system comprising computer hardware configured to perform operations comprising the methods described herein, including all embodiments thereof.

In embodiments, the disclosure provides a computer-implemented method comprising the methods described herein, including all embodiments thereof.

In embodiments, the disclosure provides computer control systems that are programmed to implement the methods of the disclosure, including all embodiments thereof. A computer system can be programmed or otherwise configured to implements methods of the disclosure, including all embodiments thereof. The computer system can be integral to implementing methods provided herein, which may be otherwise difficult to perform in the absence of the computer system. The computer system can be an electronic device of a user or a computer system that is remotely located with respect to the electronic device. The electronic device can be a mobile electronic device. As an alternative, the computer system can be a computer server.

The computer system includes a central processing unit (CPU, also “processor” and “computer processor”), which can be a single core or multi-core processor, or a plurality of processors for parallel processing. The computer system also includes memory or memory location (e.g., random-access memory, read-only memory, flash memory), electronic storage unit (e.g., hard disk), communication interface (e.g., network adapter) for communicating with one or more other systems, and peripheral devices, such as cache, other memory, data storage and/or electronic display adapters. The memory, storage unit, interface and peripheral devices are in communication with the CPU through a communication bus, such as a motherboard. The storage unit can be a data storage unit (or data repository) for storing data. The computer system can be operatively coupled to a computer network (“network”) with the aid of the communication interface. The network can be the internet, an internet and/or extranet, or an intranet and/or extranet that is in communication with the internet. The network in some cases is a telecommunication and/or data network. The network can include one or more computer servers, which can enable distributed computing, such as cloud computing. The network. in some cases with the aid of the computer system, can implement a peer-to-peer network, which may enable devices coupled to the computer system to behave as a client or a server.

The CPU can execute a sequence of machine-readable instructions, which can be embodied in a program or software. The instructions may be stored in a memory location, such as the memory. The instructions can be directed to the CPU, which can subsequently program or otherwise configure the CPU to implement methods of the present disclosure. Examples of operations performed by the CPU can include fetch, decode, execute, and writeback.

The CPU can be part of a circuit, such as an integrated circuit. One or more other components of the system can be included in the circuit. In some cases, the circuit is an application specific integrated circuit (ASIC).

The storage unit can store files, such as drivers, libraries, and saved programs. The storage unit can store user data, e.g., user preferences and user programs. The computer system in some cases can include one or more additional data storage units that are external to the computer system, such as located on a remote server that is in communication with the computer system through an intranet or the internet.

The computer system can communicate with one or more remote computer systems through the network. For instance, the computer system can communicate with a remote computer system of a user (e.g., patient, healthcare provider, or service provider). Examples of remote computer systems include personal computers (e.g., portable PC), slate or tablet PC's (e.g., Apple® iPad, Samsung® Galaxy Tab), telephones, Smart phones (e.g., Apple® iphone, Android-enabled device, Blackberry®), or personal digital assistants. The user can access the computer system via the network. Methods as described herein can be implemented by way of machine (e.g., computer processor) executable code stored on an electronic storage location of the computer system, such as, for example, on the memory or electronic storage unit. The memory can be part of a database. The machine executable or machine-readable code can be provided in the form of software. During use, the code can be executed by the processor. In embodiments, the code can be retrieved from the storage unit and stored on the memory for ready access by the processor. In embodiments, the electronic storage unit can be precluded, and machine-executable instructions are stored on memory.

The code can be pre-compiled and configured for use with a machine having a processer adapted to execute the code, or can be compiled during runtime. The code can be supplied in a programming language that can be selected to enable the code to execute in a precompiled or as-compiled fashion. Aspects of the systems and methods provided herein, such as the computer system, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk.

“Storage” media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system can include or be in communication with an electronic display that comprises a user interface (UI) for providing, for example, genetic information, such as an identification of disease-causing alleles in single individuals or groups of individuals. Examples of Ul's include, without limitation, a graphical user interface (GUI) and web-based user interface (or web interface).

Methods and systems of the present disclosure can be implemented by way of one or more algorithms. An algorithm can be implemented by way of software upon execution by the central processing unit. The algorithm can, for example, prioritize a set of two or more rare genetic variants based on a risk score of each of the two or more rare genetic variants.

Disclosed herein, in embodiments, are reports, such as wellness reports. The reports are generated using the methods and systems described herein, to provide the individual subject with results from the analyses of the genotype of the individual subject for one or more specific phenotypic traits described herein. In some cases, the reports comprise a recommendation to the individual, such as a behavior modification or product (e.g., drug) recommendation.

In embodiments, the report comprises a result from the analysis that is represented in a range (e.g., normal to high) of risk for developing or having the specific phenotypic trait of interest, which is relative to a control population. In some cases, the control population made up of individuals of the same ancestry as the individual subject. In some cases, the reference population is not ancestry-specific to the individual subject. In general, a normal result indicates that the individual subject is not predisposed to developing or having the phenotypic trait. In contrast, a high result indicates that the individual subject has a higher likelihood to develop or have the phenotypic trait, as compared to the control population. A low risk indicates that the individual subject is predisposed not to have or develop the specific phenotypic trait. A slightly high or slightly low result indicates a score between a normal score and a high or a low score, respectively.

The reports described herein, in some cases, provide product recommendations based on the specific phenotypic trait. In a non-limiting example, an individual predisposed to developing obesity (e.g., score in the 50^{th} percentile or more) would be recommended a behavior modification and/or product to prevent or slow the onset of obesity. In embodiments, the reports also comprise a hyperlink for the product that is recommended. The hyperlink will direct the individual to an online resource related to that product, such as an online commerce platform to purchase the product, or a research article or literature review article related to the specific phenotype.

Reports disclosed herein, in embodiments, provide the individual with results for multiple specific phenotypic traits, such as those described herein. For example, a single report in some cases includes results for one or more specific phenotypic traits related to one or more of disease, fitness, nutrition, and others.

The reports are formatted for delivery to the individual using any suitable method, including electronically or by mail. In embodiments, the reports are electronic reports. Electronic reports, in some cases, are formatted to transmit via a computer network to a personal electronic device of the individual (e.g., tablet, laptop, smartphone, fitness tracking device). In embodiments, the report is integrated into a mobile application on the personal electronic device. In embodiments, the App is interactive, and permits the individual to click on hyperlinks embedded within the report that automatically redirect the user to an online resource. In embodiments, the reports are encrypted or otherwise secured to protect the privacy of the individual. In embodiments, the reports are printed and mailed to the individual.

In embodiments, the software programs described herein include a web application. In light of the disclosure provided herein, those of skill in the art will recognize that a web application may utilize one or more software frameworks and one or more database systems. A web application, for example, is created upon a software framework such as Microsoft® .NET or Ruby on Rails (RoR). A web application, in embodiments, utilizes one or more database systems including, by way of non-limiting examples, relational, non-relational, feature oriented, associative, and XML database systems. Suitable relational database systems include, by way of non-limiting examples, Microsoft® SQL Server, mySQL™, and Oracle®. Those of skill in the art will also recognize that a web application may be written in one or more versions of one or more languages. In embodiments, a web application is written in one or more markup languages, presentation definition languages, client-side scripting languages, server-side coding languages, database query languages, or combinations thereof. In embodiments, a web application is written to some extent in a markup language such as Hypertext Markup Language (HTML), Extensible Hypertext Markup Language (XHTML), or extensible Markup Language (XML). In embodiments, a web application is written to some extent in a presentation definition language such as Cascading Style Sheets (CSS). In embodiments, a web application is written to some extent in a client-side scripting language such as Asynchronous Javascript and XML (AJAX), Flash® Actionscript, Javascript, or Silverlight®. In embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tel, Smalltalk, WebDNA®, or Groovy. In embodiments, a web application is written to some extent in a database query language such as Structured Query Fanguage (SQF). A web application may integrate enterprise server products such as IBM® Fotus Domino®. A web application may include a media player clement. A media player clement may utilize one or more of many suitable multimedia technologies including, by way of non-limiting examples, Adobe® Flash®, HTMF 5, Apple® QuickTime®, Microsoft® Silverlight®, Java™, and Unity®.

In embodiments, software programs described herein include a mobile application provided to a mobile digital processing device. The mobile application may be provided to a mobile digital processing device at the time it is manufactured. The mobile application may be provided to a mobile digital processing device via the computer network described herein.

A mobile application is created by techniques known to those of skill in the art using hardware, languages, and development environments known to the art. Those of skill in the art will recognize that mobile applications may be written in several languages. Suitable programming languages include, by way of non-limiting examples, C, C++, C #, Featurcive-C, Java™, Javascript, Pascal, Feature Pascal, PythonTM, Ruby, VB.NET, WMF, and XHTMF/HTMF with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Fite, .NET Compact Framework, Rhomobile, and WorkFight Mobile Platform. Other development environments may be available without cost including, by way of non-limiting examples, Fazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Android™ Market, BlackBerry® App World, App Store for Palm devices, App Catalog for webOS, Windows® Marketplace for Mobile, Ovi Store for Nokia® devices, Samsung® Apps, and Nintendo® DSi Shop.

In embodiments, the software programs described herein include a standalone application, which is a program that may be run as an independent computer process, not an add-on to an existing process, e.g., not a plug-in. Those of skill in the art will recognize that standalone applications are sometimes compiled. In embodiments, a compiler is a computer program(s) that transforms source code written in a programming language into binary feature code such as assembly language or machine code. Suitable compiled programming languages include, by way of non-limiting examples, C, C++, Featureive-C, COBOL, Delphi, Eiffel, Java™, Lisp, Perl, R, Python™, Visual Basic, and VB .NET, or combinations thereof. Compilation may be often performed, at least in part, to create an executable program. In embodiments, a computer program includes one or more executable complied applications.

Disclosed herein are software programs that, in embodiments, include a web browser plug-in. In computing, a plug-in, in embodiments, is one or more software components that add specific functionality to a larger software application. Makers of software applications may support plug-ins to enable third-party developers to create abilities which extend an application, to support casily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe® Flash® Player, Microsoft® Silverlight®, and Apple@ QuickTime®. The toolbar may comprise one or more web browser extensions, add-ins, or add-ons. The toolbar may comprise one or more explorer bars, tool bands, or desk bands. Those skilled in the art will recognize that several plug-in frameworks are available that enable development of plug-ins in various programming languages, including, by way of non-limiting examples, C++, Delphi, Java™, PHP, Python™, and VB .NET, or combinations thereof.

In embodiments, web browsers (also called internet browsers) are software applications, designed for use with network-connected digital processing devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari®, Opera Software® Opera®, and KDE Konqueror. The web browser, in embodiments, is a mobile web browser. Mobile web browsers (also called mircrobrowsers, mini-browsers, and wireless browsers) may be designed for use on mobile digital processing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser. RIM BlackBerry® Browser, Apple® Safari®, Palm@ Blazer, Palm® WebOS® Browser, Mozilla Firefox® for mobile, Microsoft® Internet Explorer® Mobile, Amazon® Kindle® Basic Web, Nokia® Browser, Opera Software® Opera® Mobile, and Sony PSP™ browser.

The medium, method, and system disclosed herein comprise one or more softwares, servers, and database modules, or use of the same. In view of the disclosure provided herein, software modules may be created by techniques known to those of skill in the art using machines, software, and languages known to the art. The software modules disclosed herein may be implemented in a multitude of ways. In embodiments, a software module comprises a file, a section of code, a programming feature, a programming structure, or combinations thereof. A software module may comprise a plurality of files, a plurality of sections of code, a plurality of programming features, a plurality of programming structures, or combinations thereof. By way of non-limiting examples, the one or more software modules comprises a web application, a mobile application, and/or a standalone application. Software modules may be in one computer program or application. Software modules may be in more than one computer program or application. Software modules may be hosted on one machine. Software modules may be hosted on more than one machine. Software modules may be hosted on cloud computing platforms. Software modules may be hosted on one or more machines in one location. Software modules may be hosted on one or more machines in more than one location.

The medium, method, and system disclosed herein comprise one or more databases, such as the phenotypic and/or genotypic-associated database described herein, or use of the same. In embodiments, the database are used for rare genetic variants, and optionally common genetic variants.

Those of skill in the art will recognize that many databases are suitable for storage and retrieval of information. Suitable databases include, by way of non-limiting examples, relational databases, non-relational databases, feature oriented databases, feature databases, entity-relationship model databases, associative databases, and XML databases. In embodiments, a database is internet-based. In embodiments, a database is web-based. In embodiments, a database is cloud computing-based. A database may be based on one or more local computer storage devices.

The methods, systems, and media described herein, are configured to be performed in one or more facilities at one or more locations. Facility locations are not limited by country and include any country or territory. In embodiments, one or more steps of a method herein are performed in a different country than another step of the method. In embodiments, one or more steps for obtaining a sample are performed in a different country than one or more steps for analyzing a genotype of a sample. In embodiments, one or more method steps involving a computer system are performed in a different country than another step of the methods provided herein. In embodiments, data processing and analyses are performed in a different country or location than one or more steps of the methods described herein. In embodiments, one or more articles, products, or data are transferred from one or more of the facilities to one or more different facilities for analysis or further analysis. An article includes, but is not limited to, one or more components obtained from a sample of a subject and any article or product disclosed herein as an article or product. Data includes, but is not limited to, information regarding genotype and any data produced by the methods disclosed hercin. In embodiments of the methods and systems described herein, the analysis is performed, and a subsequent data transmission step will convey or transmit the results of the analysis.

In embodiments, any step of any method described herein is performed by a software program or module on a computer. In embodiments, data from any step of any method described herein is transferred to and from facilities located within the same or different countries, including analysis performed in one facility in a particular location and the data shipped to another location or directly to an individual in the same or a different country. In embodiments, data from any step of any method described herein is transferred to and/or received from a facility located within the same or different countries, including analysis of a data input, such as cellular material, performed in one facility in a particular location and corresponding data transmitted to another location, or directly to an individual, such as data related to the diagnosis, prognosis, responsiveness to therapy, or the like, in the same or different location or country.

Embodiments disclosed herein provide one or more non-transitory computer readable storage media encoded with a software program including instructions executable by the operating system. In embodiments, software encoded includes one or more software programs described herein. In embodiments, a computer readable storage medium is a tangible component of a computing device. In embodiments, a computer readable storage medium is optionally removable from a computing device. In embodiments, a computer readable storage medium includes, by way of non-limiting examples, CD-ROMs, DVDs, flash memory devices, solid state memory, magnetic disk drives, magnetic tape drives, optical disk drives, cloud computing systems and services, and the like. In embodiments, the program and instructions are permanently, substantially permanently, semi-permanently, or non-transitorily encoded on the media.

### Phenotypes

As the skilled artisan will appreciate, the presence of rare genetic variants within the genome of the individual can be determined by methods known in the art. In embodiments, the presence of rare genetic variants within the genome of the individual subject can be determined by analyzing a biological sample obtained from an individual subject for the presence of the rare genetic variants. Methods for analyzing the genome of an individual subject and methods of detecting common genetic variants and rare genetic variants in an individual subject are known in the art and can be used in the methods described therein.

In embodiments of the methods described herein, the phenotype is a pulmonary disease, an inflammatory disease, cancer, an autoimmune disease, a neurodegenerative disease, a psychiatric disease, a substance use disorder, or a cardiovascular disease. In embodiments, the phenotype is a pulmonary disease. In embodiments, the phenotype is an inflammatory disease. In embodiments, the phenotype is cancer. In embodiments, the phenotype is an autoimmune disease. In embodiments, the phenotype is a neurodegenerative disease. In embodiments, the phenotype is a psychiatric disease. In embodiments, the phenotype is a substance use disorder. In embodiments, the phenotype is a cardiovascular disease. In embodiments, the phenotype is obesity, breast cancer, or type 2 diabetes.

The term “pulmonary disease” refers to lung disorders characterized by difficulty breathing, coughing, airway discomfort and inflammation, increased mucus, and/or pulmonary fibrosis. Examples of pulmonary diseases include lung cancer, cystic fibrosis, asthma, chronic obstructive Pulmonary Disease, bronchitis, emphysema, bronchiectasis, pulmonary edema, pulmonary fibrosis, sarcoidosis, pulmonary hypertension, pneumonia, tuberculosis, Interstitial Pulmonary Fibrosis,

Interstitial Lung Disease, Acute Interstitial Pneumonia, Respiratory Bronchiolitis-associated Interstitial Lung Disease, Desquamative Interstitial Pneumonia, Non-Specific Interstitial Pneumonia, Idiopathic Interstitial Pneumonia, Bronchiolitis obliterans, with Organizing Pneumonia, restrictive lung disease, or pleurisy.

The term “inflammatory disease” refers to a disease or condition characterized by aberrant inflammation (e.g., an increased level of inflammation compared to a control such as a healthy person not suffering from a disease). Examples of inflammatory diseases include autoimmune diseases, arthritis, rheumatoid arthritis, psoriatic arthritis, juvenile idiopathic arthritis, multiple sclerosis, systemic lupus erythematosus, myasthenia gravis, juvenile onset diabetes, diabetes mellitus type 1, graft-versus-host discase, Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, ichthyosis, Graves ophthalmopathy, inflammatory bowel disease, Addison's disease, vitiligo, asthma, allergic asthma, acne vulgaris, celiac disease, chronic prostatitis, inflammatory bowel disease, pelvic inflammatory disease, reperfusion injury, ischemia reperfusion injury, stroke, sarcoidosis, transplant rejection, interstitial cystitis, atherosclerosis, scleroderma, and atopic dermatitis.

The term “cancer” refers to all types of cancer, neoplasm or malignant tumors found in mammals (e.g., humans), including leukemias, lymphomas, carcinomas and sarcomas. Exemplary cancers include brain cancer, glioma, glioblastoma, neuroblastoma, prostate cancer, colorectal cancer, pancreatic cancer, medulloblastoma, melanoma, cervical cancer, gastric cancer, ovarian cancer, lung cancer, cancer of the head, Hodgkin's Disease, and Non-Hodgkin's Lymphomas. Exemplary cancers that may be treated with a compound or method provided herein include cancer of the thyroid, endocrine system, brain, breast, cervix, colon, head & neck, liver, kidney, lung, ovary, pancreas, rectum, stomach, and uterus. Additional examples include, thyroid carcinoma, cholangiocarcinoma, pancreatic adenocarcinoma, skin cutaneous melanoma, colon adenocarcinoma, rectum adenocarcinoma, stomach adenocarcinoma, esophageal carcinoma, head and neck squamous cell carcinoma, breast invasive carcinoma, lung adenocarcinoma, lung squamous cell carcinoma, non-small cell lung carcinoma, mesothelioma, multiple myeloma, neuroblastoma, glioma, glioblastoma multiforme, ovarian cancer, rhabdomyosarcoma, primary thrombocytosis, primary macroglobulinemia, primary brain tumors, malignant pancreatic insulanoma, malignant carcinoid, urinary bladder cancer, premalignant skin lesions, testicular cancer, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

The term “autoimmune disease” refers to a disease or condition in which a subject's immune system has an aberrant immune response against a substance that does not normally elicit an immune response in a healthy subject. Examples of autoimmune diseases include acute disseminated encephalomyelitis (ADEM), acute necrotizing hemorrhagic leukoencephalitis, Addison's disease, agammaglobulinemia, alopecia arcata, amyloidosis, ankylosing spondylitis, Anti-GBM/Anti-TBM nephritis, antiphospholipid syndrome (APS), autoimmune angioedema, autoimmune aplastic anemia, autoimmune dysautonomia, autoimmune hepatitis, autoimmune hyperlipidemia, autoimmune immunodeficiency, autoimmune inner ear disease (AIED), autoimmune myocarditis, autoimmune oophoritis, autoimmune pancreatitis, autoimmune retinopathy, autoimmune thrombocytopenia purpura (ATP), autoimmune thyroid disease, autoimmune urticaria, axonal or neuronal neuropathies, balo disease, Behcet's disease, bullous pemphigoid, cardiomyopathy, castleman disease, celiac disease, chagas disease, chronic fatigue syndrome, chronic inflammatory demyelinating polyneuropathy (CIDP), chronic recurrent multifocal ostomyelitis (CRMO), Churg-Strauss syndrome, cicatricial pemphigoid/benign mucosal pemphigoid, Crohn's disease, Cogans syndrome, cold agglutinin disease, congenital heart block, coxsackie myocarditis, CREST disease, essential mixed cryoglobulinemia, demyelinating neuropathies, dermatitis herpetiformis, dermatomyositis, Devic's disease (neuromyelitis optica), discoid lupus, Dressler's syndrome, endometriosis, cosinophilic esophagitis, cosinophilic fasciitis, erythema nodosum, experimental allergic encephalomyelitis, Evans syndrome, fibromyalgia, fibrosing alveolitis, giant cell arteritis (temporal arteritis), giant cell myocarditis, glomerulonephritis, Goodpasture's syndrome, granulomatosis with polyangiitis, Graves' disease, Guillain-Barre syndrome, Hashimoto's encephalitis, Hashimoto's thyroiditis, Hemolytic anemia, Henoch-Schonlein purpura, Herpes gestationis, Hypogammaglobulinemia, Idiopathic thrombocytopeniaurpura (ITP), IgA nephropathy, IgG4-related sclerosing disease, Immunoregulatory lipoproteins, Inclusion body myositis, Interstitial cystitis, Juvenile arthritis, Juvenile diabetes (Type 1 diabetes), Juvenile myositis, Kawasaki syndrome, Lambert-Eaton syndrome, Leukocytoclastic vasculitis, lichen planus, lichen sclerosus, Igneous conjunctivitis, linear IgA disease (LAD), lupus (SLE), lyme disease, chronic, Meniere's disease, Microscopic polyangiitis, Mixed connective tissue disease (MCTD), Mooren's ulcer, Mucha- Habermann disease, multiple sclerosis, myasthenia gravis, myositis, narcolepsy, neuromyelitis optica (Devic's), neutropenia, ocular cicatricial pemphigoid, optic neuritis, palindromic rheumatism, pediatric autoimmune neuropsychiatric disorders associated with streptococcus, parancoplastic cerebellar degeneration, paroxysmal nocturnal hemoglobinuria, Parry Romberg syndrome, Parsonnage-Turner syndrome, pars planitis, Pemphigus, peripheral neuropathy, perivenous encephalomyelitis, pernicious anemia, POEMS syndrome, polyarteritis nodosa, Type I, II, & III autoimmune polyglandular syndromes, polymyalgia rheumatica, polymyositis, postmyocardial infarction syndrome, postpericardiotomy syndrome,progesterone dermatitis, primary biliary cirrhosis, primary sclerosing cholangitis, psoriasis, psoriatic arthritis, Idiopathic pulmonary fibrosis, pyoderma gangrenosum, Pure red cell aplasia, Raynauds phenomenon, Reactive Arthritis, reflex sympathetic dystrophy, Reiter's syndrome, relapsing polychondritis, restless legs syndrome, Retroperitoneal fibrosis, rheumatic fever, rheumatoid arthritis, sarcoidosis, Schmidt syndrome, Scleritis, scleroderma, Sjogren's syndrome, sperm & testicular autoimmunity, Stiff person syndrome, subacute bacterial endocarditis (SBE), Susac's syndrome, sympathetic ophthalmia, Takayasu's arteritis, temporal arteritis/Giant cell arteritis, thrombocytopeniapurpura (TTP), Tolosa-Hunt syndrome, transverse myelitis, type 1 diabetes, ulcerative colitis, undifferentiated connective tissue disease, uveitis, vasculitis, vesiculobullous dermatosis, vitiligo, or Wegener's granulomatosis.

The term “neurodegenerative disease” refers to a disease or condition in which the function of a subject's nervous system becomes impaired. Examples of neurodegenerative diseases include Alexander's disease, Alper's disease, Alzheimer's disease, Amyotrophic lateral sclerosis, Ataxia telangiectasia, Batten disease, Bovine spongiform encephalopathy, Canavan disease, chronic fatigue syndrome, Cockayne syndrome, Corticobasal degeneration, Creutzfeldt-Jakob disease, frontotemporal dementia, Gerstmann-Sträussler-Scheinker syndrome, Huntington's disease, HIV-associated dementia, Kennedy's disease, Krabbe's disease, kuru, Lewy body dementia, Machado-Joseph disease, Multiple sclerosis, Multiple System Atrophy, myalgic encephalomyelitis, Narcolepsy, Neuroborreliosis, Parkinson's disease, Pelizacus-Merzbacher Disease, Pick's disease, Primary lateral sclerosis, Prion diseases, Refsum's disease, Sandhoff's disease, Schilder's disease, Subacute combined degeneration of spinal cord secondary to Pernicious Anaemia, Schizophrenia, Spinocerebellar ataxia, Spinal muscular atrophy, Steele-Richardson-Olszewski disease, progressive supranuclear palsy, or tabes dorsalis.

The term “psychiatric disease” is used in accordance with its plain and ordinary meaning in the art. In embodiments, the psychiatric disease is an autism spectrum disorder, attention-deficit hyperactivity disorder, a mood disorder, schizophrenia, depression, mania, bipolar disorder, an eating disorder, anxiety, a panic disorder, obsessive-compulsive disorder, a phobia, a psychotic disorder, a personality disorder, or post-traumatic stress disorder.

The term “substance use disorder” refers disorders that involve the use, dependence, withdrawal, or addiction to substances. In embodiments, the substance use disorder is opioid use disorder, alcohol use disorder, tobacco use disorder, and the like. The substance use disorder can involve alcohol, marijuana, hallucinogens (e.g., PCP, LSD), inhalants (e.g., gluc, paint thinners), opioids (e.g., prescription, heroin), sedatives, hypnotics, anxiolytics, stimulants (e.g., cocaine, methamphetamine), and tobacco.

The term “cardiovascular disease” is used in accordance with its plain and ordinary meaning in the art. In embodiments, cardiovascular diseases include stroke, heart failure, hypertension, hypertensive heart disease, myocardial infarction, angina pectoris, tachycardia, cardiomyopathy, rheumatic heart disease, cardiomyopathy, heart arrhythmia, congenital heart disease, valvular heart disease, carditis, aortic aneurysms, peripheral artery disease, thromboembolic disease, and venous thrombosis.

The term “obesity” or “overweight” refers to a disorder involving excessive body fat that increases the risk of health problems (e.g., type 2 diabetes, cardiovascular disorders). Obesity is a chronic, relapsing, multifactorial, neurobehavioral disease, wherein an increase in body fat promotes adipose tissue dysfunction and abnormal fat mass physical forces, resulting in adverse metabolic, biomechanical, and psychosocial health consequences. In embodiments, obesity refers to a subject having a BMI of 25 or more. In embodiments, obesity refers to a subject having a BMI of 30 or more. In embodiments, obesity refers to a subject having a BMI of 35 or more. In embodiments, obesity refers to a subject having a BMI of 40 or more. In embodiments, obesity refers to a male having an abdominal circumference of 40 inches or more. In embodiments, obesity refers to a male having 21% body fat or more. In embodiments, obesity refers to a male having 25% body fat or more. In embodiments, obesity refers to a female having an abdominal circumference of 35 inches or more. In embodiments, obesity refers to a female having 31% body fat or more. In embodiments, obesity refers to a female having 33% body fat or more.

The term “breast cancer” refers to a cancer that forms in the cells of the breast. In embodiments, the breast cancer is ductal carcinoma. In embodiments, the breast cancer is lobular carcinoma. In embodiments, the breast cancer is triple-negative breast cancer. In embodiments, the breast cancer is triple-positive breast cancer. In embodiments, the breast cancer is inflammatory breast cancer. In embodiments, the breast cancer is ER-positive. In embodiments, the breast cancer is PR-positive. In embodiments, the breast cancer is hormone-receptor positive. In embodiments, the breast cancer is hormone-receptor negative. In embodiments, the breast cancer is HER2-positive.

The term “type 2 diabetes” is a chronic condition that develops when the body becomes resistant to insulin or when the pancreas is unable to produce enough insulin. In embodiments, a subject with type 2 diabetes has an AlC of 5.7% or more. In embodiments, a subject with type 2 diabetes has an AIC of 6.5% or more. In embodiments, a subject with type 2 diabetes has an AlC of 7% or more. In embodiments, a subject with type 2 diabetes has an fasting blood sugar level of at least 100 mg/dl. In embodiments, a subject with type 2 diabetes has an fasting blood sugar level of at least 126 mg/dl.

In embodiments of the methods described herein, the phenotype is obesity. In embodiments, the phenotype is obesity and plurality of different rare genetic variants comprises at least 2 different rare genetic variants set forth in Table A. In embodiments, the phenotype is obesity and plurality of different rare genetic variants comprises at least 10 different rare genetic variants set forth in Table A. In embodiments, the phenotype is obesity and plurality of different rare genetic variants comprises at least 20 different rare genetic variants set forth in Table A. In embodiments, the phenotype is obesity and plurality of different rare genetic variants comprises at least 30 different rare genetic variants set forth in Table A. In embodiments, the phenotype is obesity and plurality of different rare genetic variants comprises at least 40 different rare genetic variants set forth in Table A. In embodiments, the phenotype is obesity and plurality of different rare genetic variants comprises at least 50 different rare genetic variants set forth in Table A. In embodiments, the phenotype is obesity and plurality of different rare genetic variants comprises at least 100 different rare genetic variants set forth in Table A. In embodiments, the phenotype is obesity and plurality of different rare genetic variants comprises at least 200 different rare genetic variants set forth in Table A. In embodiments, the phenotype is obesity and plurality of different rare genetic variants comprises at least 300 different rare genetic variants set forth in Table A. In embodiments, the phenotype is obesity and plurality of different rare genetic variants comprises at least 400 different rare genetic variants set forth in Table A. In embodiments, the phenotype is obesity and plurality of different rare genetic variants comprises at least 500 different rare genetic variants set forth in Table A. In embodiments, the phenotype is obesity and plurality of different rare genetic variants comprises at least 1,000 different rare genetic variants set forth in Table A. In embodiments, the phenotype is obesity and plurality of different rare genetic variants comprises at least 5,000 different rare genetic variants set forth in Table A. In embodiments, the phenotype is obesity and plurality of different rare genetic variants comprises at least 10,000 different rare genetic variants set forth in Table A. In embodiments, the phenotype is obesity and plurality of different rare genetic variants comprises at least 20,000 different rare genetic variants set forth in Table A. In embodiments, the phenotype is obesity and plurality of different rare genetic variants comprises at least 30,000 different rare genetic variants set forth in Table A. In embodiments, the phenotype is obesity and plurality of different rare genetic variants comprises at least 40,000 different rare genetic variants set forth in Table A. In embodiments, the phenotype is obesity and plurality of different rare genetic variants comprises at least 50,000 different rare genetic variants set forth in Table A. In embodiments, the phenotype is obesity and plurality of different rare genetic variants comprises at least 60,000 different rare genetic variants set forth in Table A. In embodiments, the phenotype is obesity and plurality of different rare genetic variants comprises at least 70,000 different rare genetic variants set forth in Table A. In embodiments, the phenotype is obesity and plurality of different rare genetic variants comprises at least 80,000 different rare genetic variants set forth in Table A.

In embodiments of the methods described herein, the phenotype is breast cancer. In embodiments, the phenotype is breast cancer and plurality of different rare genetic variants comprises at least 2 different rare genetic variants set forth in Table B. In embodiments, the phenotype is breast cancer and plurality of different rare genetic variants comprises at least 10 different rare genetic variants set forth in Table B. In embodiments, the phenotype is breast cancer and plurality of different rare genetic variants comprises at least 20 different rare genetic variants set forth in Table B. In embodiments, the phenotype is breast cancer and plurality of different rare genetic variants comprises at least 30 different rare genetic variants set forth in Table B. In embodiments, the phenotype is breast cancer and plurality of different rare genetic variants comprises at least 40 different rare genetic variants set forth in Table B. In embodiments, the phenotype is breast cancer and plurality of different rare genetic variants comprises at least 50 different rare genetic variants set forth in Table B. In embodiments, the phenotype is breast cancer and plurality of different rare genetic variants comprises at least 100 different rare genetic variants set forth in Table B. In embodiments, the phenotype is breast cancer and plurality of different rare genetic variants comprises at least 200 different rare genetic variants set forth in Table B. In embodiments, the phenotype is breast cancer and plurality of different rare genetic variants comprises at least 300 different rare genetic variants set forth in Table B. In embodiments, the phenotype is breast cancer and plurality of different rare genetic variants comprises at least 400 different rare genetic variants set forth in Table B. In embodiments, the phenotype is breast cancer and plurality of different rare genetic variants comprises at least 500 different rare genetic variants set forth in Table B. In embodiments, the phenotype is breast cancer and plurality of different rare genetic variants comprises at least 1,000 different rare genetic variants set forth in Table B. In embodiments, the phenotype is breast cancer and plurality of different rare genetic variants comprises at least 5,000 different rare genetic variants set forth in Table B. In embodiments, the phenotype is breast cancer and plurality of different rare genetic variants comprises at least 10,000 different rare genetic variants set forth in Table B. In embodiments, the phenotype is breast cancer and plurality of different rare genetic variants comprises at least 15,000 different rare genetic variants set forth in Table B.

In embodiments of the methods described herein, the phenotype is type 2 diabetes. In embodiments, the phenotype is type 2 diabetes and plurality of different rare genetic variants comprises at least 2 different rare genetic variants set forth in Table C. In embodiments, the phenotype is type 2 diabetes and plurality of different rare genetic variants comprises at least 10 different rare genetic variants set forth in Table C. In embodiments, the phenotype is type 2 diabetes and plurality of different rare genetic variants comprises at least 20 different rare genetic variants set forth in Table C. In embodiments, the phenotype is type 2 diabetes and plurality of different rare genetic variants comprises at least 30 different rare genetic variants set forth in Table C. In embodiments, the phenotype is type 2 diabetes and plurality of different rare genetic variants comprises at least 40 different rare genetic variants set forth in Table C. In embodiments, the phenotype is type 2 diabetes and plurality of different rare genetic variants comprises at least 50 different rare genetic variants set forth in Table C. In embodiments, the phenotype is type 2 diabetes and plurality of different rare genetic variants comprises at least 100 different rare genetic variants set forth in Table C. In embodiments, the phenotype is type 2 diabetes and plurality of different rare genetic variants comprises at least 200 different rare genetic variants set forth in Table C. In embodiments, the phenotype is type 2 diabetes and plurality of different rare genetic variants comprises at least 300 different rare genetic variants set forth in Table C. In embodiments, the phenotype is type 2 diabetes and plurality of different rare genetic variants comprises at least 400 different rare genetic variants set forth in Table C. In embodiments, the phenotype is type 2 diabetes and plurality of different rare genetic variants comprises at least 500 different rare genetic variants set forth in Table C. In embodiments, the phenotype is type 2 diabetes and plurality of different rare genetic variants comprises at least 1,000 different rare genetic variants set forth in Table C. In embodiments, the phenotype is type 2 diabetes and plurality of different rare genetic variants comprises at least 5,000 different rare genetic variants set forth in Table C. In embodiments, the phenotype is type 2 diabetes and plurality of different rare genetic variants comprises at least 10,000 different rare genetic variants set forth in Table C. In embodiments, the phenotype is type 2 diabetes and plurality of different rare genetic variants comprises at least 20,000 different rare genetic variants set forth in Table C. In embodiments, the phenotype is type 2 diabetes and plurality of different rare genetic variants comprises at least 30,000 different rare genetic variants set forth in Table C. In embodiments, the phenotype is type 2 diabetes and plurality of different rare genetic variants comprises at least 40,000 different rare genetic variants set forth in Table C. In embodiments, the phenotype is type 2 diabetes and plurality of different rare genetic variants comprises at least 50,000 different rare genetic variants set forth in Table C. In embodiments, the phenotype is type 2 diabetes and plurality of different rare genetic variants comprises at least 60,000 different rare genetic variants set forth in Table C. In embodiments, the phenotype is type 2 diabetes and plurality of different rare genetic variants comprises at least 70,000 different rare genetic variants set forth in Table C. In embodiments, the phenotype is type 2 diabetes and plurality of different rare genetic variants comprises at least 80,000 different rare genetic variants set forth in Table C.

In embodiments, the methods described herein further comprising treating the individual subject having the phenotype or monitoring the individual subject having the phenotype (e.g., via diagnostic screening. The terms “treating”, or “treatment” refers to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, may include prevention of an injury, pathology, condition, or discase. In embodiments, treating is preventing. In embodiments, treating does not include preventing.

“Treating” or “treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms, fully or partially remove the disease's underlying cause, shorten a disease's duration, or do a combination of these things.

“Treating” and “treatment” as used herein include prophylactic treatment. Treatment methods include administering to a subject a therapeutically effective amount of an active agent. The administering step may consist of a single administration or may include a series of administrations. he length of the treatment period depends on a variety of factors, such as the severity of the condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In embodiments, chronic administration may be required. For example, the compositions are administered to the subject in an amount and for a duration sufficient to treat the patient. In embodiments, the treating or treatment is no prophylactic treatment.

Aspects disclosed herein provide methods and systems for administering (e.g., treating, recommending) to an individual a behavioral modification related to a specific phenotypic trait, based at least in part, on the presence of the plurality of different rare genetic variants within the genome of the individual. In embodiments, a plurality of administrations (e.g., treatments, recommendations) of behavior modifications are provided to the individual. In embodiments, a survey of the individual is provided by the individual comprising questions related to the specific phenotypic trait of interest. In embodiments, the behavior modifications are based on the presence of the plurality of different rare genetic variants within the genome of the individual for the trait, and the answers to the questions received from the individual subject. In embodiments, the behavior modification comprises increasing, reducing, or avoiding an activity. Non-limiting examples of activities include, but are not limited to, comprising a physical exercise, ingestion of a substance (e.g., supplement or drug), exposure to a product (e.g., fumes, toxins, irritants, and the like), usage of a product (e.g., skin care product, hair care product, nail care product, and the like), a diet, a lifestyle, sleep, and consumption (e.g., consumption of alcohol, a drug, caffeine, an allergen, a food or category of foods). In embodiments, the behavior modification comprises an activity to remedy or prevent the specific phenotypic trait (for e.g., engaging or not engaging in an activity that serves as a cause or a correlative to the occurrence of the specific phenotypic trait).

The present disclosure provides, by way of non-limiting examples, various recommendations of behavior modifications related to the specific phenotypic traits described herein. In embodiments, an individual with plurality of different rare genetic variants indicating an increased likelihood for obesity, as compared to a subject population, is recommended to engage in an activity to remedy and/or prevent obesity (e.g., engaging in exercise, engaging in nutritious counsel, taking anti-obesity drugs). In embodiments, an individual with plurality of different rare genetic variants indicating an increased likelihood for alcoholism, as compared to a subject population, is recommended to avoid consumption of alcohol, or to reduce alcohol consumption.

When the phenotype is obesity, the individual subject can be monitored for obesity. In embodiments, the individual subject with an obesity phenotype can be administered an effective amount of a suitable medication. The treatment can be determined or adjusted according to the risk of obesity. The treatment can comprise administering an effective amount of orlistat, phentermine/topiramate, buproprion/naltrexone, or a GLP-1 agonist, such as liraglutide. In embodiments, the treatment may comprise an endoscopic procedure or bariatric surgery (e.g., gastric bypass surgery, adjustable gastric banding, biliopancreatic diversion with duodenal switch, or a gastric sleeve).

When the phenotype is breast cancer, the individual subject can be monitored for breast cancer. In embodiments, the individual subject with a breast cancer phenotype can be administered an effective amount of an anti-cancer agent. The treatment can be determined or adjusted according to the risk of breast cancer. The treatment can comprise administering drugs for treating or preventing breast cancer, e.g., raloxifene hydrochloride, tamoxifen citrate, or a combination thereof. Alternatively, or additionally, the treatment can comprise administering drugs for treating breast cancer. Exemplary drugs include Abemaciclib, Abraxane (Paclitaxel Albumin-stabilized Nanoparticle Formulation), Ado-rastuzumab Emtansine, Afinitor (Everolimus), Anastrozole, Aredia (Pamidronate Disodium), Anastrozole, Aromasin (Exemestane), Capecitabine, Cyclophosphamide, Docetaxel, Doxorubicin Hydrochloride, Ellence (Epirubicin Hydrochloride), Epirubicin Hydrochloride, Eribulin Mesylate, Everolimus, Exemestane, 5-FU (Fluorouracil Injection), Fareston (Toremifene), Faslodex (Fulvestrant), Femara (Letrozole), Fluorouracil Injection, Fulvestrant, Gemcitabine Hydrochloride, Gemcitabine Hydrochloride, Goserelin Acetate, Halaven (Eribulin Mesylate), Herceptin (Trastuzumab), Ibrance (Palbociclib), Ixabepilone, Ixempra (Ixabepilone), Kadcyla (Ado-Trastuzumab Emtansinc), (Ribociclib), Lapatinib Ditosylate, Letrozole, Lynparza (Olaparib), Megestrol Acetate, Methotrexate, Neratinib Malcate, Neratinib Malcate, Olaparib, Paclitaxel, Paclitaxel Albumin-stabilized Nanoparticle Formulation, Palbociclib, Pamidronate Disodium, Perj cta (Pertuzumab), Pertuzumab, Ribociclib, Tamoxifen Citrate, Paclitaxel, Docetaxel, Thiotepa, Toremifene, Trastuzumab, Trexall (Methotrexate), Lapatinib Ditosylate, Verzenio (Abemaciclib), Vinblastine Sulfate, Capecitabine, Zoladex (Goserelin Acetate), or any combination thereof. When the phenotype is type 2 diabetes, the individual subject can be monitored for diabetes. In embodiments, the individual subject with a type 2 diabetes phenotype can be administered an effective amount of a diabetes agent. In embodiments, the diabetes agent is metformin, insulin, thiazolidinediones (glitazones), biguanides, meglitinides, DPP-4 inhibitors, GLP-1 agonists, sodium-glucose transporter 2 (SGLT2) inhibitors, alpha-glucosidase inhibitors, bile acid sequestrants, incretin-based therapies, sulfonylurcas, amylin analogs, or a combination of two or more thereof. In sembodiments, the biguanide is metformin. In embodiments, the meglitinide is repaglinide or nateglinide. In embodiments, the sulfonylurea is chlorpropamide, glipizide, glyburide, or glimepiride. In embodiments, the thiazolidinedione is rosiglitazone or pioglitazone. In embodiments, the DPP-4 inhibitor is Sitagliptin, saxagliptin, linagliptin, or alogliptin. In embodiments, the SGLT2 inhibitors is Canagliflozin or dapagliflozin. In embodiments, the alpha-glucosidase inhibitor is acarbose or miglitol. In embodiments, the bile acid sequestrate is colesevelam. In embodiments, the GLP-1 agonist is exenatide, lixisenatide, liraglutide, albiglutide, dulaglutide, or semaglutide.

In embodiments, diseases described herein can be treated with gene therapy/genome editing and/or a nucleic acid vector used in a gene therapy. In embodiments, one or more target locus within the subject's genomic DNA is targeted and modified. A treatment method comprises gene editing tools available in the art, e.g., CRISPR, zinc finger nucleases, meganuceases, where a target DNA locus, e.g., a gene of interest, is modified to create a mutation in the gene product, e.g., a protein or enzyme, with reduced activity or no activity (loss-of-function mutation). In embodiments, vectors can comprise viral vector, e.g., retroviruses, adenoviruses, adeno-associated viruses, and lentiviruses.

### Kits and Articles of Manufacture

Also disclosed herein, in embodiments, are kits useful for to detect the genotypes (e.g., rare genetic variants, common genetic variants) described herein. In embodiments, the kits disclosed herein may be used to predict whether an individual has, or will develop, a specific phenotype trait. In embodiments, the kits are useful to diagnose or prognose a disease or condition in an individual subject. In embodiments, the kits are useful for selecting an individual subject for treatment. In embodiments, the kit is accompanied by a product recommendation, such as a supplement, or over-the-counter medication. In embodiments, the kit is accompanied by a recommendation to consult a physician or medical healthcare professional.

In embodiments, the kit comprises components to perform methods of detecting the genotypes (e.g., rare genetic variants) described herein. Kits comprise an assemblage of materials or components. In embodiments, the kits contain all of the components necessary and/or sufficient to perform an assay for detecting the genotypes (e.g., rare genetic variants), including all controls, directions for performing assays, and any necessary software for analysis and presentation of results. In embodiments, the kits are suitable for assays such as PCR, and qPCR. In embodiments, the kit comprises a genotyping chip that can be used at the point of need. The exact nature of the components configured in the kit depends on its intended purpose.

Instructions for use may be included in the kit. Optionally, the kit also contains other useful components, such as, diluents, buffers, pharmaceutically acceptable carriers, syringes, catheters, applicators, pipetting or measuring tools, bandaging materials, or other useful paraphernalia. The materials or components assembled in the kit can be provided to the practitioner stored in any convenient and suitable ways that preserve their operability and utility. For example, the components can be in dissolved, dehydrated, or lyophilized form; they can be provided at room, refrigerated or frozen temperatures. The components are typically contained in suitable packaging material(s). As employed herein, packaging material refers to one or more physical structures used to house the contents of the kit, such as compositions and the like. The packaging material is constructed by well-known methods, preferably to provide a sterile, contaminant-free environment. The packaging materials employed in the kit are those customarily utilized in gene expression assays and in the administration of treatments. As used herein, the term package refers to a suitable solid matrix or material such as glass, plastic, paper, foil, and the like, capable of holding the individual kit components. Thus, for example, a package can be a glass vial or prefilled syringes used to contain suitable quantities of the pharmaceutical composition. The packaging material has an external label which indicates the contents and/or purpose of the kit and its components.

### Embodiments

Embodiment 1. A method to enhance existing polygenic risk scores by accounting for rare genetic or de novo variants; wherein the method identifies rare variants to include in a polygenic risk model by selecting variants with large effects on molecular phenotypes or endophenotypes (i.e., gene expression.

Embodiment 2. The method of Embodiment 1 where the effect sizes or number of rare variants can predict risk of a complex trait or disease.

Embodiment 3. The method of Embodiment 1 where rare variants to integrate in PRS are select by either effect size or p-value.

Embodiment 4. The method of Embodiment 1 where the effect sizes or number of rare variants combined with the polygenetic risk score.

Embodiment 5. The method of Embodiment 1 where an individual's rare variants can modify only specific PRS for that same individual.

Embodiment 6. The method of Embodiment 1 that integrates different classes and categories of large-effect rare variants including structural variants, copy number variants and insertion-deletion polymorphism into a polygenic risk model.

Embodiment 7. The method of Embodiment 1 that integrates a subset of large-effect rare variants for genes with molecular etiology matches into a polygenic risk model.

Embodiment 8. Genes detected through GWAS/eQTL colocalization being a subset of highly actionable loci for a complex trait where expression mediates risk. Subsequent large effect rare variants may then be identified that modify expression. This subset has higher priors on modifying the complex trait due to shared molecular etiology at the locus. “Matching molecular etiology” approach that extends to all molecular phenotypes or endophenotypes.

Embodiment 9. The method of Embodiment 1 that integrates large effect rare variants into polygenic risk scores based on the heritability of the trait. Highly heritable traits will have larger improvements by integrating rare large-effect variants.

Embodiment 10. A database of rare large-effect variants for integration with polygenic risk scores in individuals where molecular phenotype or endophenotype measurements have not been obtained.

Embodiment 11. The method of Embodiment 1 where rare variants with large phenotypic effects are catalogued for use in polygenic risk models.

Embodiment 12. The method of Embodiment 1 applied to human individuals.

Embodiment 13. The method of Embodiment 1 is used to modify predictions that lead to intervention, treatments or enhance diagnosis and diagnostic strategies.

Embodiment 14. The method of Embodiment 1 where identification of rare variants is through sequencing.

Embodiment 15. The method of Embodiment 1 using molecular or endophenotype measurements from pathologically-relevant cell types.

Embodiment 16. The method of Embodiment 1 using a combination of molecular or endophenotype measurements.

Embodiment 17. The method of Embodiment 1 which is not limited to rare variant detection in specific cell types alone.

Embodiment 18. A computer program product comprising machine-readable medium storing instructions that, when executed by at least one programmable processor, cause the at least one programmable processor to perform the method of any one of Embodiments 1 to 17.

Embodiment 19. A system comprising computer hardware configured to perform the method of any one of Embodiments 1 to 17.

Embodiment 20. The system of Embodiment 19, wherein the computer hardware comprises a programmable processor; and machine-readable medium storing instructions that, when executed by the processor, cause the at least one programmable processor to perform at least one of the methods.

### EXAMPLES

Given the known large effects of rare variants linked to expression outliers-and that these variants are not currently included in existing PRS-Applicants sought to test where this subset of rare variants can aid in explaining instances where an individual's phenotype deviates substantially from their phenotype as predicted by PRS. Presented herein is an approach that summarizes the phenotypic effects of an increasing burden of rare variants associated with outlier gene expression in GTEx and also present in UKBB. Data presented herein are for BMI and obesity given the growing public health emergency of severe obesity in the US and around the world, the availability of high-quality publicly-available PRS for BMI, known polygenicity and sample size considerations.

Polygenic risk scores (PRS) aim to quantify the contribution of multiple genetic loci to a complex trait. However, existing PRS estimate genetic liability using common genetic variants excluding the impact of rare variants. To assess the impact of rare variants on complex traits and PRS predictions, Applicant identified rare variants in individuals with outlier gene expression from GTEx that were also present as rare variants in the UK Biobank (UKBB). Across multiple UKBB GWAS, larger complex trait effects were observed for expression outlier rare variants compared to control variants, increasing with the degree of outlier severity. Applicant further observed large deviations from the PRS-predicted phenotype for BMI in the UKBB for carriers of expression outlier rare variants; for example, individuals classified as “low-risk” but in the top percentile of outlier rare variant burden had a six-fold higher rate of severe obesity. Findings were replicated using data from the NHLBI Trans-Omics for Precision Medicine (TOPMed) biobank and Million Veterans Project and demonstrate that multiple PRS will significantly benefit from the inclusion of rare genetic variants.

### Example 1

Integration of large-effect expression variants redefines polygenic risk prediction.

Results: Identification of large-effect, rare expression variants

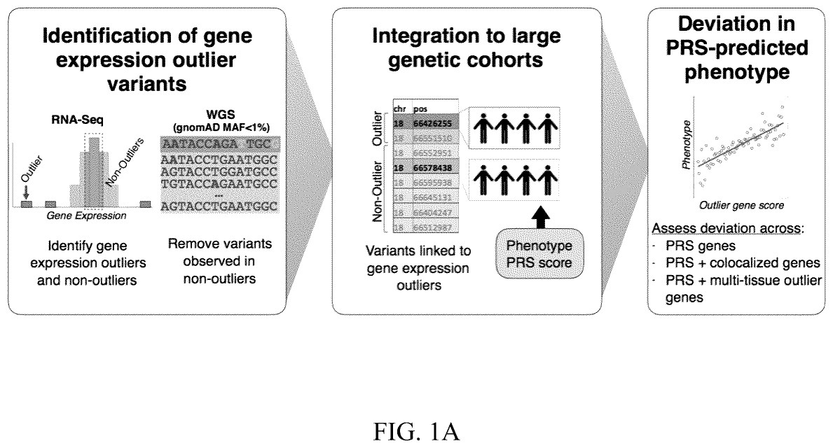

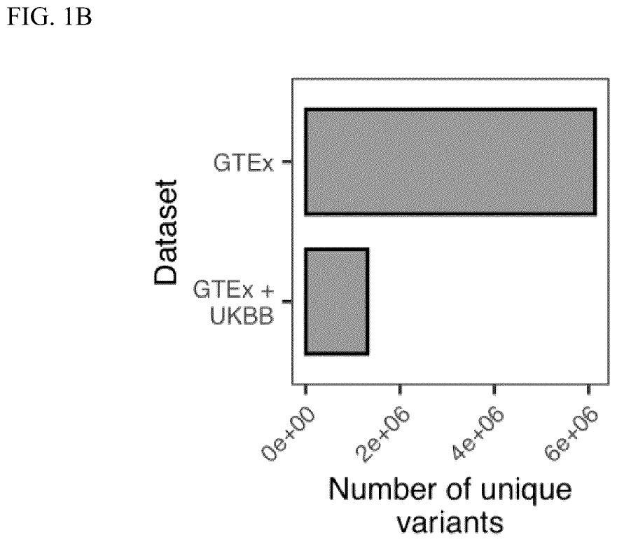

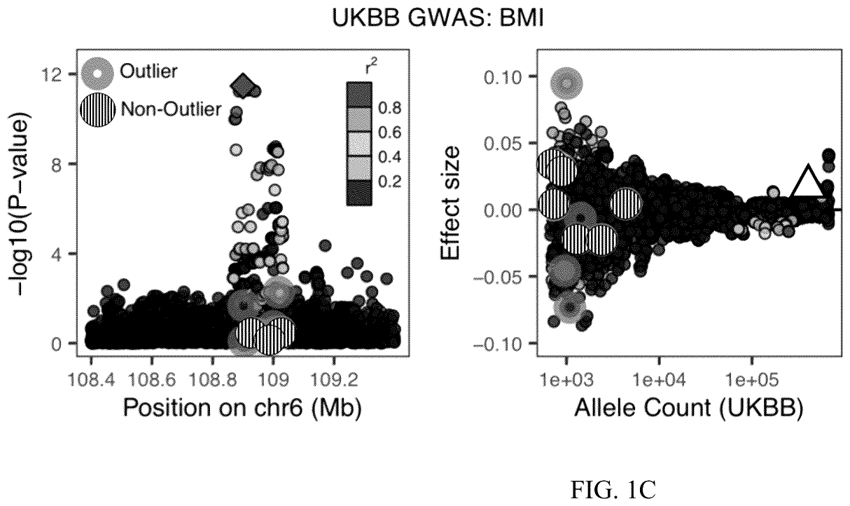

To identify rare variants linked to gene expression outliers that could also be tested for their effects on complex traits, Applicants intersected the set of variants with gnomAD MAF >0 and <1% identified in GTEx v7 with high-quality imputed variants in the UKBB (FIG. 1A). From a starting set of 6,134,805 unique rare variants, 1,307,023 (21.3%) variants were identified also within the UKBB (FIG. 1B). From this intersecting set, a tabulation was performed across all GTEx individuals to isolate the subset of rare variants present in gene expression outlier individuals. This process was conducted in two ways; “top-outlier” where only rare variants from the most extreme outlier individual(s) (maximum of two individuals per gene), and “all outliers” where all rare variants from individuals with abs(Z)>2 were included. The intersecting list of rare variants found in both outlier and non-outlier individuals were subsequently removed. Variants were then linked to a specific gene if they fell within the gene body or +/−10 Kb (n genes: “top-outlier”=3,732; “all-outliers”=15,095).

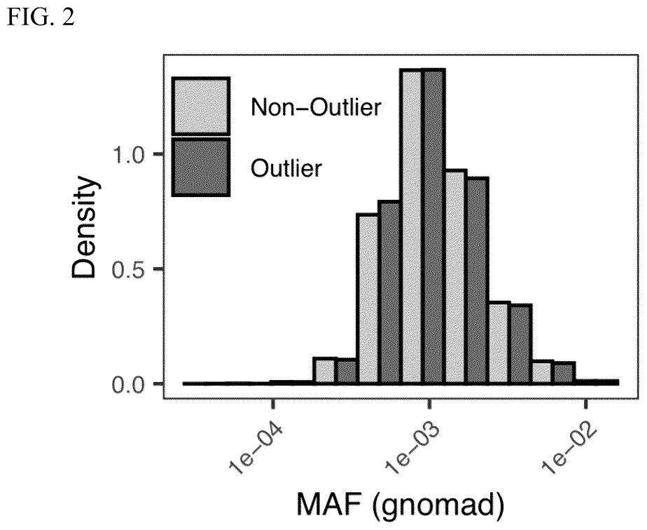

Applicants further defined a corresponding set of non-outlier/control variants, matched on both the gnomAD MAF and CADD scores of outlier variants (FIG. 2).

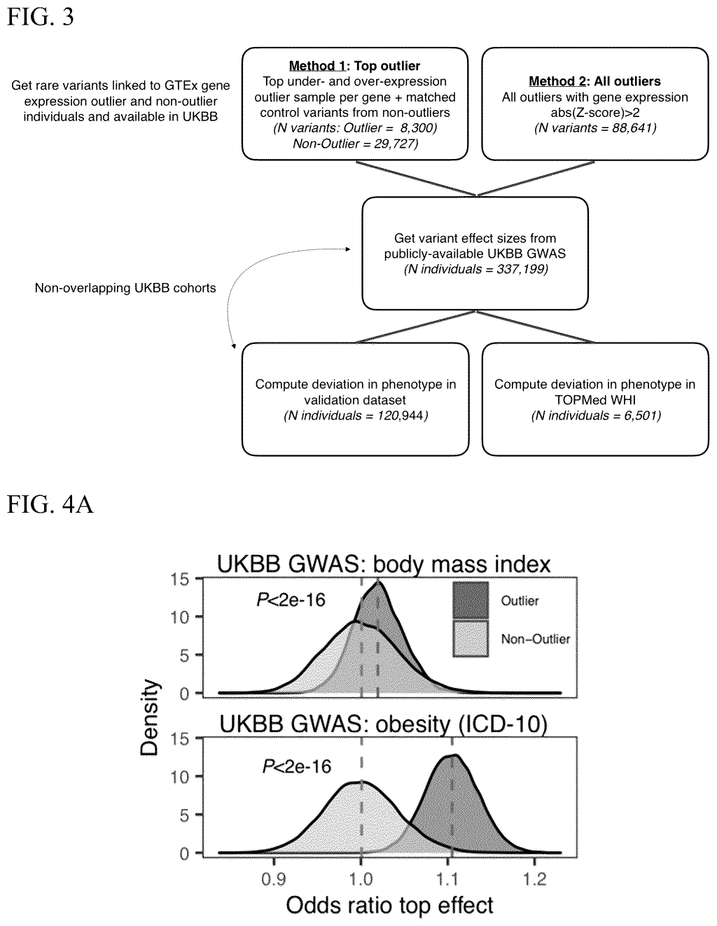

Applicants observed that individuals were often carriers for multiple outlier rare variants. Considering a sample cohort of individuals from UKBB (N=120,944) (FIG. 3), each individual had an average of 23 (“top-outlier”) and 304 (“all-outlier”) outlier variants. To evaluate if these variants cumulatively were biased in effect direction (i.e., risk or protective) for a highly polygenic trait, Applicants assessed UKBB BMI GWAS effect directions and observed no significant differences. On average, individuals carried 11 potential protective rare variants and 12 potential risk variants using the “top-outlier” approach.

Large-effect, rare expression variants impact BMI and obesity in UK Biobank

To evaluate if rare expression outlier-associated variants had greater effect sizes than matched control variants, Applicants focused on BMI and obesity GWAS from the UKBB. Applicants first noticed that some outlier variants in isolation had the potential for significantly large effects; for example, an outlier variant linked to the gene FOXO3 was observed with effect size rank of 1/3059 in a 1 Mb locus, and among the top 0.07% of effect sizes overall, across all variants measured across the UKBB for BMI (FIG. 1C).

To systematically assess whether outlier variants had higher effect sizes than control variants, Applicants performed a permutation test (N permutations=10,000) using outlier (“top-outlier”; n variants =8,272) and matched control variants (n variants=29,659) that fall within 10 kb of any PRS variant to assess how often randomly-drawn outlier variants had larger effect sizes than control variants. For BMI, a mean odds ratio of 1.02 was observed when comparing outlier vs. non-outlier variants, and a mean odds ratio of 1 when comparing non-outlier variants to themselves (Wilcox test, P<1e-16). For obesity (ICD-10 E66), an increased mean odds ratio of 1.1 (Wilcox test, P<1e-16) was observed (FIG. 4A). When increasing the outlier expression Z-score threshold, progressively larger odds ratios (mean odds ratio: abs(Z)>4=1.28; abs(Z)>6=1.58) were observed, but not when comparing non-outlier variants only (mean odds ratio: abs(Z)>4=1; abs(Z)>6=1) (Wilcox test, P<1e-16 for both comparisons) (FIG. 4B). Applicants replicated the permutation test findings using a subset of rare variants available in the Million Veteran Project (MVP) BMI GWAS (N variants: outlier=4,955; non-outlier=18,145), and observed similar results (mean odds ratio: outlier vs. non-outlier=1.05; non-outlier only=1; P <1c-16) (FIG. 4C). Applicants further directly compared effect sizes between outlier and control variants and observed significantly increased effect sizes for outlier variants that increased with outlier Z-score thresholds (FIG. 4D; Ansari test, P<1e-16 for all comparisons).

To identify the impact of outlier variants in an existing, publicly-available BMI PRS, Applicants used data from Khera et al. (2019)¹. Applicants first obtained gnomAD AF for PRS variants, and observed that these variants have a mean gnomAD AF=0.49 (SD=0.29) (FIG. 4E). Plotting GWAS effect sizes by UKBB allele count for an example locus (gene FOXO3) further illustrates that PRS variants tend to be common variants with small effects (FIG. 4F). Applicants calculated PRS for each individual in UKBB and observed the expected gradients in mean BMI and weight increasing by PRS deciles (FIGS. 5A-5B). Applicants then used a linear regression model to assess change in BMI given an individual's PRS, sex, age, first ten components of genetic ancestry, genotyping array, and a score that quantifies the total outlier-variant burden per individual, computed by subtracting total protective from total risk outlier-variants collapsed to gene-level (FIG. 6). This score is referred to as the independent outlier gene count (IOGC) score henceforth. Applicants observed significant coefficient estimates for 10/15 features in the model, including IOGC score (linear regression r=0.015, P=7e-07) (FIG. 7).

Applicants sought to clarify whether variants identified from outlier individuals in GTEx were driving downstream effects on phenotype as measured in UKBB in excess of what might be observed by selecting random subsets of non-outlier rare variants. This was investigated by first selecting random subsets of the matched non-outlier variants (matching the number of outlier variants across permutations, N variants =8,272; N permutations=10,000), observing that the IOGC beta estimate when using outlier variants exceeds that which would be expected based on random subsets of non-outlier variants (mean IOGC non-outlier =0.0075; empirical P=0.0012) (FIG. 8A), validating the findings of the permutation test described in the previous section. Next Applicants used information on multi-tissue outliers; that is, the number of tissues an outlier is observed in—increasing the confidence of causal outlier expression effects. For a multi-tissue outlier threshold of >=1 (i.e. all outlier variants), Applicants observe a mean change in BMI of 0.015 kg/m² per unit change in IOGC score (linear regression, P=7e-07), whereas increasing this threshold to >=10 tissues results in a greater than 5-fold increase in mean change in BMI to 0.08 kg/m² per unit change in IOGC score (linear regression, P=0.04) (FIG. 4G). Using a permutation test of non-outlier variants (N permutations =10,000), Applicants again observe that outlier effects exceed that which would be expected using random subsets of rare variants (FIG. 8B).

Applicants investigated whether the severity of outlier gene expression affected change in BMI. At increasingly more-stringent Z-score thresholds, it was observed that mean change in BMI also increased (FIG. 4H) (abs(Z) 2-3, linear regression r=0.008 (P<1e-16); abs(Z) 3-4, linear regression r=0.009 (P=2c-06); abs(Z) 4-5, linear regression r=0.014 (P=9e-04). Comparing variants identified in outlier genes with Z-score between abs(Z) 2-3 with abs(Z) 4-5, a 75% increase was observed in mean change in BMI per unit change in IOGC score.

Applicants leveraged GTEx cis-eQTL summary statistics to further check for concordance in GWAS effect direction between cis-eQTL and outlier variants matched on slope (e.g., positive cis-cQTL slope and over-expression outlier). Applicants stratified results by cis-eQTL variant GWAS p-value and outlier-associated variant Z-score, observing that variants identified in more-severe (by Z-score) expression outliers have overall better concordance in GWAS effect direction with cis-eQTL variants, across genes. For example, at a cis-cQTL variant GWAS P-value cutoff <=1e-06, Applicants observed 50, 96, and 100% concordance for variants passing absolute Z-score thresholds of 2, 3, and 4, respectively (FIGS. 9A-9B).

Carriers of multiple large-effect, rare expression variants have increased risk.

From the analyses presented above, rare variants linked to outlier individuals in GTEx had larger effects on BMI and rates of obesity, independent of PRS, and this effect is modulated by properties of outlier effects (i.e. multi-tissue outliers, outlier Z-score severity). Applicants next sought to understand the magnitude of deviation from cohort-average BMI and obesity associated with outlier rare variant burden. For this analysis outlier-associated variants identified were used using the all-outlier method-this increases the range of IOGC scores that can be interrogated (range: top-outlier =-19:20; all-outliers =-67:69).

Applicants calculated the mean rate of change across percentiles of IOGC score using a linear regression model, adjusting for PRS, age, sex, first ten principal components of ancestry, and genotyping array. Each increment in IOGC score percentile bin is associated with a mean rate of change in BMI of 0.05 kg/m² (linear regression, P <le-16); comparing bottom and top 0.05% percentiles, this results in a difference in mean BMI of 0.74 kg/m² (FIG. 10A). In the regression model, Applicants tested for an interaction between PRS and IOGC score and observed no significant effect.

Rates of obesity (BMI>=30 kg/m²) and severe obesity (BMI >=40 kg/m²) for individuals in the extreme 0.5% IOGC score percentiles deviate from the average rates of the full cohort overall: obesity: 0.5^{th} percentile =20.8%; 99.5^{th} percentile=26.6%; average overall: 24.5% (logistic regression, P=5.3c-14); severe obesity: 0.5^{th} percentile=1%; 99.5^{th} percentile =3.5%; average overall: 1.9% (logistic regression, P=0.001) (FIG. 10B). Applicants also tested for risk of being underweight (BMI<18.5 kg/m²) and found an inverse relationship (i.e. lower IOGC score percentile increases risk of being underweight) (logistic regression, P=0.003).

Individuals in extreme IOGC score percentiles further differed in their age of onset of obesity and high blood pressure diagnosis (where high blood pressure is used as a proxy for hypertension). For diagnosis of obesity, individuals in IOGC score percentile <=1%, mean age of onset obesity=59.41, whereas individuals in IOGC score percentile>=99%, mean age of onset=56.95, a difference of in age of onset of 2.46 years (Wilcox test, P=0.03). For high blood pressure diagnosis, individuals in IOGC score percentile<=1%, mean age of onset =53.04, whereas individuals in IOGC score percentile>=99%, mean age of onset=50.44, a difference of 2.6 years (Wilcox test, P=0.004) (FIG. 10C).

Thus, Applicants have established a baseline expected deviation from cohort-average BMI and rates of obesity associated with outlier-variant burden in a subset of genes overlapping PRS variants. Applicants next integrated GTEx gene colocalization results¹⁷. Running the linear regression model for IOGC score with increasing numbers of colocalized genes (N colocalized genes>=1, 2, 3 or 4), observed an increase in mean change in BMI associated was observed with each percentile change in IOGC score (linear regression r=0.07 kg/m² (P=5c-11); 0.07 kg/m² (P=0.0001); 0.13 kg/m² (P=0.0006); and 0.2 kg/m² (P=0.01), respectively).

Applicants also observed that effects of outlier rare variants can manifest from childhood. A subset of individuals (N=55,126) was identified who provided self-reported information on being “plumper” or “thinner” than average at age 10 (UKBB data field #1687). Applicants tested the association of IOGC score with childhood body size using a logistic regression model (where the response was coded as 0=″thinner“, 1=″plumper”). The model was adjusted for PRS, age, sex, and first ten principal components of ancestry. For each unit change in IOGC score, Applicants observed an increase in the odds of having a “plumper” comparative body size at 10 of 1.001 (logisitc regression, P=3e-09).

Rare variants increase stratification of polygenic risk prediction.

Applicants showed in the linear regression model that IOGC score is a significant predictor of BMI, independent from a range of covariates including PRS. Applicants repeated the analysis in explicit subsets of individuals, namely individuals stratified to PRS bin 1 (“low-risk”) and bin 10 (“high-risk”), and observed broadly similar linear regression coefficients for unit change in IOGC score percentile (PRS bin 1: r=0.06 (P=3e-05); PRS bin 10: r=0.11 (P=7e-07) (FIG. 10D). Applicants tested for an interaction between PRS and IOGC score and observed no significant effect.

Applicants next sought to understand the potential magnitude of deviation in PRS-predicted rate of severe obesity (BMI >=40 kg/m²) associated with extreme IOGC score. Applicants first stratified individuals by PRS-“low-risk” (PRS bin 1), “intermediate risk” (PRS bin 2-9), and “high-risk” (PRS bin 10), and further subset increasingly stringent percentiles of IOGC score (Table 1). Empirical P- values were computed using a permutation test (N permutations =10,000) to understand how likely the rates of severe obesity are observed across random subsets of individuals from within the same PRS groups. From this analysis Applicants observed, for example, that “low-risk” individuals (PRS bin 1) in the 99th percentile of IOGC score have a rate of severe obesity approaching the average rate for PRS bin 10 (4.55%, P=0.0009), a greater than 6-fold increase in PRS-predicted severe obesity.

TABLE 1
Rates of severe obesity as a function of PRS and IOGC score (*
P-values are calculated empirically across 10,000 permutations)

| | IOGC percentile (mean IOGC) | | | | | | | | |
|---|---|---|---|---|---|---|---|---|---|
| | <=0.25% | <=0.5% | <=1% | <=10% | | >=90% | >=99% | >=99.5% | >=99.75% |
| | (−47) | (−43) | (−40) | (−26) | PRS only | (31) | (46) | (49) | (52) |
| PRS | 0% | 0% | 0% | 0.66% | 0.67% | 1.01% | 4.55% | 6.35% | 6.06% |
| bin 1 | (n: 45) | (n: 84) | (n: 171) | (n: 1,351) | (n: 12,094) | (n: 992) | (n: 110) | (n: 63) | (n: 33) |
| | P = NS | P = NS | P = NS | P = NS | | P = NS | P = 0.0009 | P = 0.0006 | P = 0.02 |
| PRS | 0% | 1.72% | 2.65% | 4.08% | 4.99% | 5.28% | 7.63% | 10.35% | 16.67% |
| bin 10 | (n: 27) | (n: 58) | (n: 113) | (n: 1152) | (n: 12,043) | (n: 1137) | (n: 118) | (n: 58) | (n: 30) |
| | P = NS | P = NS | P = NS | P = NS | | P = NS | P = NS | P = NS | P = 0.02 |

Integration of rare variants into PRS: replication in TOPMed WHI

Applicants replicated their findings using TOPMed WHI data, subset to individuals with European ancestry and with genetic and phenotypic data available ( =6,501). Applicants constructed a linear regression model including PRS, first ten principal components of ancestry, age, and IOGC score (sex is not included since TOPMed WHI is an all-female cohort). IOGC score is again a significant predictor of BMI (mean change in BMI per quantile of IOGC score: linear regression r=0.13 kg/m², P=0.03) (FIG. 10E). Although explicitly tested in the model, Applicants compared the PRS of individuals in the 10th and 90th percentile of IOGC score and observed no significant differences in PRS for these two groups (FIG. 11). In the regression model, an interaction between PRS and IOGC score was tested again and observed no significant effect. Similar to before, rates of obesity (BMI>=30 kg/m²) and severe obesity (BMI >=40 kg/m²) for individuals in the 10^{th} and 90^{th} percentiles for IOGC score deviated from the average rates of the full cohort overall: obesity: 10^{th} percentile=31.03%; 90^{th} percentile =35.30%; average overall: 33.49% (FIG. 12); severe obesity: 10^{th} percentile =3.67%; 90^{th} percentile =5.43%; average overall: 4.59% (FIG. 10E).

Subsetting by multi-tissue outlier-associated variants (N tissues >=10), Applicants again observed a significant effect of IOGC score independent from PRS, age and genetic ancestry (linear regression, P=0.03); notably, a per unit change in IOGC score is associated with a 15-fold increase in BMI compared with selecting all outlier-associated variants (linear regression r: N tissue >=10: 0.11; N tissue >=1: 0.007). Further highlighting the independence of IOGC score from PRS, risk of obesity and severe obesity among individuals within PRS bin 10 (“high-risk”) can vary substantially from average for individuals in the 10th and 90th percentile of IOGC score (FIG. 10F).

Rare variants impact polygenic risk prediction across multiple traits and diseases.

The focus of the study was on BMI and associated rates of obesity, but it was observed that the same approach can be applied to many other traits and diseases. For example, using a publically-available PRS for type-2 diabetes, Applicants observed a deviation from PRS-predicted mean incidence of diabetes associated with an increasing burden of outlier-associated variants (logistic regression, r=1.01, P=0.03) (FIG. 13A). Looking at age of T2D onset in a cohort defined as “high-risk” by PRS (PRS Z-score >1), Applicants observe a difference in mean age of onset of 4.04 years (wilcox test, P=0.02), comparing individuals in the 10^{th} and 90^{th} percentiles of IOGC score among this PRS high-risk group (FIG. 13B).

To quantify differences in effect sizes of outlier-associated variants across diverse traits and disease, the same permutation test described earlier was repeated, utilizing all outlier-and non-outlier variants identified using the “top-outlier” method across 2,419 traits and diseases released in UKBB Phase 1 GWAS (N permutations =1,000). Applicants observed a mean odds ratio of 1.05 (SD =0.06) across all disease and traits when comparing outlier vs. non-outlier variants, and a mean odds ratio of 1 (SD=0.002) for non-outlier variants only (Wilcox test, P <2e-16) (FIG. 13C). Increasing the outlier Z-score threshold, Applicants observed an increasing trend for observing outlier variants with top effect sizes (mean odds (SD): abs(Z-score)>4=1.10 (0.14); abs(Z-score)>6 =1.17 (0.25) (Wilcox test, P <1e-16 both comparisons). No difference was observed in odds ratios when comparing non-outlier variants only.

Across different GWAS meta-categories (cancer, illnesses, physical traits, cause of death), Applicants observed the same overall trend for observing outlier variants with top GWAS effect sizes, increasing with Z-score threshold (P<2e-16 for all outlier vs. non-outlier comparisons) (FIG. 13D). For example, for breast cancer (ICD-10: C50, Malignant neoplasms of breast), Applicants observed odds ratios 1.02, 1.11, 1.25 for abs(Z-score)>2, 4 and 6, respectively. Applicants also expected some GWAS traits to not be sensitive to SNPs linked to gene outlier effects. This was explored by manually selecting several what was termed “non-genetic” GWAS traits and observed no difference in the distribution of odds ratios comparing outlier vs. non-outlier variants and non-outlier variants only (FIG. 14).

### Discussion

Applicants have demonstrated that a high burden of rare, outlier-linked variants can lead to substantial deviations in PRS-predicted phenotype. Some limitations may be considered. Rare, expression outlier-linked variants were isolated in GTEx v7, but-given that this cohort is around 450 individuals—it is certain that many large-effect variants impacting expression remain to be identified. Future large-scale RNA-sequencing studies and catalogues of outlier-associated rare variants will be useful for this task. Furthermore, Applicants could only recover a subset of outlier-associated rare variants in UKBB, due to limitations in imputation; with WGS (release by UKBB), Applicants recover more outlier-associated variants from GTEx in UKBB. Imputation also means Applicants were limited in terms of frequency spectra that could be integrated-singleton and ultra-rare variants will likely have even larger effects and impacts on the IOGC score¹⁸⁻²⁰.

Another potential limitation is possible false positives among the set of outlier-linked variants. It can be assumed that because a variant is observed in an outlier only (i.e., not found in a non-outlier individual) that a causal variant is isolated (i.e., a variant driving the observed expression dysregulation), but this could be easily observed by chance. Expanded sample sizes would help to control the false positive rate. Furthermore, GWAS effect size estimates were ignored for rare variants when calculating IOGC, as these estimates are associated with large standard errors, instead using effect direction only (i.e., risk/protective). Thus, tests with larger cohort sizes mean these effect size estimates are used in the calculation of IOGC score, potentially with greater discriminatory power to assess deviation in PRS-predicted phenotype.

The exemplary experiments described herein offers a baseline of phenotypic effects of rare, large-effect variants and shows considerable impact in aiding to predict individual phenotypes. Applicants have shown that multi-tissue outlier and gene variance both increase the deviation in PRS-predicted phenotype. Further work may integrate other data modalities (e.g., variant annotation from VEP). Overall, this work has implications for the continued implementation of rare genetic variants, optionally in combination with PRS, in standard clinical care.

### Example 2

Processed WGS variant data was obtained from GTEx v7 (see Resource Availability). Using the software bedtools²¹ (—window flag), variants were linked to genes if falling within the gene body, 10 Kb upstream of transcription start, or 10 Kb downstream of the transcription end site. Using the software Vcfanno²², SNP variants were intersected with gnomAD (version r2.0.2)²³ and CADD²⁴ databases to obtain the minor allele frequency and CADD score, respectively, for each SNP variant. Minor allele frequencies were not ancestry-specific. Non-SNP variants were discarded. SNPs were retained if the gnomAD MAF fell in the range 0<MAF<1%. Multi-allelic SNPs were removed (multi-allelic in gnomAD). Finally, SNPs were required to have been directly measured or imputed in UKBB Phase 1 GWAS.

Processed RNA-sequencing data was obtained from GTEx v7 (see Resource Availability). To identify GTEx outlier gene expression samples, normalized gene expression values (FPKM) were processed across all GTEx v7 tissues, limited to autosomal genes annotated as protein coding or long non-coding RNA genes in GENCODE v19. A minimum expression filter was applied per gene (>=10 individuals with FPKM >0.1 and read count >6); genes not passing this filter were removed. Expression values were PEER²⁵ factor corrected (using 15 factors for tissues with <=150 samples, 30 for tissues with <=250 samples, and 35 for tissues with >250 samples), then scaled and centered to generate expression Z-scores. Individuals exhibiting global patterns of outlier gene expression for a given tissue were removed from the final corrected expression matrix for that tissue. Global outlier is defined as any individual who has the most-extreme absolute Z-score of corrected gene expression in 100 or more genes in a given tissue at an outlier cutoff of abs(Z-score)>2.

### UK Biobank Data

UKBB Phase 1 GWAS summary statistics were downloaded from the Neale Lab server (available at the World Wide Web nealelab.is/uk-biobank). Summary statistics for each GTEx outlier and non-outlier variant were joined on chromosome, position, ref, and alt columns, using hg19 coordinates. All other phenotypic and genotypic data were sourced from the data instance approved under UKBB application #24983 (see Data Availability). Individual-level phenotypes for weight (UKBB data field #21002), body mass index (UKBB data field #21001) and diabetes (UKBB data field #2443) were downloaded from the relevant phenotype file. For weight and BMI, Applicants averaged (using the median) over all observations per-individual for those with multiple observations for the same phenotype. Imputed and directly measured genotypes for all variants used in this study were extracted from the genotyping callset version 3. Additional phenotypic and demographic data used included:

age, sex, principal components, genotyping array (all included in the UKBB sample QC file); age of onset of diagnosis (obesity (UKBB data field #130792), high blood pressure (UKBB data field #2966), diabetes (UKBB data field #2976)); and comparative body size at age 10 (UKBB data field #1687).

TOPMed Women's Health Initiative (WHI) data

The full TOPMed WHI cohort was first subset to self-reported European ancestry only (race code ‘5’ in file WHI.phv00078450.v6.p3.cl.txt). Individual-level weight and BMI measurements were obtained from the file phs000200.v11.pht001019.v6.p3.c1.f80_rell.HMB-IRB.txt.gz. The average (median) was found for individuals with multiple observations of the same phenotype. Genotypes were obtained from whole genome sequencing data available in the archive phg001146.v1.TOPMed_WGS_WHI.genotype-calls-vcf.c1.HMB-IRB.tar. BED files were created using the software plink (version 2.0)²⁶. TOPMed WHI bed files are in hg38 assembly; Applicants used the software CrossMap²⁷ to convert genome coordinates from hg19 to hg38 assemblies for the purposes of measuring GTEx outlier and non-outlier variants among TOPMed WHI individuals and computing a polygenic risk score.

Genotype principal components Applicants computed using a random selection of common variants (N=50,000) available in UKBB; Applicants chose to leverage UK Biobank allele count information to define a set of high-confidence common variants (UKBB minor allele count>50,000), given the increased sample size of UKBB compared with TOPMed WHI. Genotypes were extracted using plink (version 2.0)[ref]. To create the input matrix for computed principal components, the genotypes of each extract variant was imported and checked for variance and the percentage of missing genotypes;

variants with zero variants and/or >1% missingness were removed. For variants with >0 and <=1% genotype missingness, missing genotypes were replaced by the mode for that particular variant. Principal components were computed using the software flashpca²⁸.

Million Veteran Project (MVP) GWAS for body mass index

DNA extracted from participants' blood was genotyped using a customized Affymetrix Axiom® biobank array, the MVP 1.0 Genotyping Array. The array was enriched for both common and rare genetic variants of clinical significance in different ethnic backgrounds. Quality-control procedures used to assign ancestry, remove low-quality samples and variants, and perform genotype imputation to the 1000 Genomes reference panel were previously described²⁹. Individuals related more than second degree Cousins were excluded.

Applicants recently conducted HARE (Harmonized ancestry and Race/Ethnicity) analysis using race/ethnicity information from MVP participants³⁰. Genotyped MVP participants are assigned into one of the four HARE groups (Hispanics, non-Hispanics White, non-Hispanics Black, and non-Hispanics Asian) and “Other”. The analysis is based on a machine learning algorithm, which integrates race/ethnicity information from MVP baseline survey and high-density genetic variation data. Trans-ethnic, and ethnicity-specific principal component analyses were performed using flashPCA²⁸. BMI was calculated as average BMI using all measurements within a three-year window around the date of MVP enrollment (i.e., 1.5 years before/after the date of enrollment), excluding height measurements that were >3 inches or weight measurements >60 pounds from the average of each participant.

Genetic association with BMI in the MVP cohort was examined among 217,980 non-Hispanic White participants. BMI was stratified by sex and adjusted for age, age-squared, and the top ten genotype-derived principal components in a linear regression model. The resulting residuals were transformed to approximate normality using inverse normal scores. Imputed and directly measured genetic variants were tested for association with the inverse normal transformed residuals of BMI through linear regression assuming an additive genetic model.

Isolating rare variants observed in GTEx gene expression outliers and non-outliers.

Rare variants occurring in gene expression outlier individuals are identified using two methods; namely, top-outlier and all-outliers. Both approaches start with genetic and transcriptomic data processed as detailed above (“GTEx v7 genetic and transcriptomic data”). Using the top-outlier approach, variants were aggregated across all individuals and subset to gnomAD MAF >0 and <1%.

This list was then tabulated to obtain a count of unique individuals with each variant; any variant observed in >1 individual was removed. As a further filtering step, variants were retained only if they were included in UKBB Phase 1 GWAS. To link variants to expression outliers, Applicants identified for each tissue the individuals with the least or most expression per gene (i.e., under-expression outlier and over-expression outlier), removing any results falling below a pre-refined Z-score threshold of abs(Z-score) <2. Applicants also defined, for each tissue and gene, a set of individuals with non-outlier gene expression (defined as abs(Z-score)<1). Non-outlier variants were filtered to match the CADD score (within a window +/−5) of any outlier variants for each tissue/gene/outlier direction triple; this is important for genes with both an under-expression and over-expression outlier, as subsequent permutation testing uses outlier and non-outlier variants matched on a CADD score window. Variants identified in outliers in >=1 tissue were ignored when identifying matching non-outlier variants; in this way, a putatively causal large-effect expression variant (in any number of tissues) would not be counted in both outlier and non-outlier variant sets. For the all-outliers method, Applicants removed any outlier variant also identified in >=1 non-outlier individual; this differs from the top-outlier method, in which outlier variants identified in any other individual (regardless of outlier status) are removed. Applicants did not define a matching set of non-outlier variants in the all-outliers method, due to run-time constraints in the computational pipeline developed for this study. For both methods, the number of tissues was recorded in which each outlier variant was identified (for variants identified in >1 tissue, Applicants refer to these as multi-tissue outlier variants).

### GWAS Effect Size Permutation Test

Applicants performed a permutation test to study differences in GWAS effect sizes for GTEx outlier and non-outlier variants. This test was repeated for two independent GWAS cohorts: UK Biobank and Million Veterans Project. For each GWAS, the input data is a file containing outlier and non-outlier variants with associated GWAS effect size (beta), linked outlier gene, GTEx sample ID, outlier direction (under-expression/over-expression), and outlier tissue. Additionally, for GWAS of traits and disease where Applicants also run a separate test after integrating PRS information, subsetting genes to those linked to any outlier variant falling within 10 Kb of a PRS variant. To define a set of outlier and non-outlier variants, Applicants first subset outlier variants using a defined absolute Z-score of outlier gene expression, then find the intersection (using tissue, gene, and outlier direction) between the outlier variants that pass the Z-score thresholds and the matched non-outlier controls. This step ensures Applicants have sufficient data to randomly select exactly one outlier and non-outlier variant per tissue/gene/outlier direction triple (referred to as an outlier triple), per permutation. The permutation test is based on the results of the top-outlier methods (see previous section); therefore, there is exactly one outlier individual per outlier triple. However, for non-outlier variants, there can be matched variants identified in >1 unique individual. Applicant subset randomly to one non-outlier individual per outlier triple, then randomly select exactly one outlier and non-outlier variant per outlier triple. For each outlier triple, Applicants then count which variant is associated with the greater GWAS absolute effect size (outlier/non-outlier); this information can then be summarized in a contingency table, which is then used as the input to compute an odds ratio. Applicants repeated this analysis for a file containing non-outlier variants only, which follows the same method described above, comparing two randomly chosen non-outlier variants per outlier triple, for outlier triples with non-outlier variants from >=2 unique non-outlier individuals.

Calculating polygenic risk scores.

Applicants computed polygenic risk scores (PRS) for the UKBB and TOPMed WHI cohorts in this study. Two publicly available PRS were used (see Resource Availability): body mass index (Khera.ct.al_GPS_BMI_Cell_2019.txt.zip); and type-2 diabetes (Type2Diabetes_PRS_LDpred_rho0.01_v3.txt). Scores were calculated using the software plink (version 2.0) [ref] (—score flag). PRS variant coordinates were first converted to hg38 assembly using CrossMap (27) for calculating PRS scores in the TOPMed WHI cohort. Scores were calculated separately for each chromosome, then summed per individual and scaled to generate Z-scores.

### Colocalization Summary Statistics

Applicants downloaded GTEx v7 colocalization summary statistics from LocusCompare (see Resource Availability). Applicants subset summary statistics to GWAS traits matching the terms “BMI”, “Extreme BMI” or “Overweight”, across any GTEx tissue and with a colocalization CLPP score >=0.1 (N genes =202). Colocalized gene counts were computed for each individual in the UKBB cohort but cross-referencing the vector of outlier genes with the colocalization gene list, per individual. The effect of IOGC on BMI for increasing burden of outliers in colocalized genes was re-computed in a linear regression model adjusting for PRS, age, sex, first ten principal components of ancestry, and genotyping array.

GTEx eQTL to assess concordance in GWAS effect direction between eQTL and outlier variants.

GTEx eQTL summary statistics were downloaded and filtered on P-value (using column “pval_nominal”) with threshold P<1e-18. Remaining variants were linked to their GWAS effect size (i.e. protective or risk). For genes with >1 eQTL passing the P-value threshold, the variant with the smallest UKBB GWAS P-value was retained (this step was computed separately for each GTEx tissuc). Applicants used a majority-rule approach to assign a single, high-quality consensus GWAS effect direction per gene, based on the median slope estimate and GWAS effect direction across all top eQTL variants per tissue that passed the two P-value filtering steps. For example, if a given gene consisted of 10 eQTL risk variants and 5 protective risk variants, the gene would be assigned a “risk” label. Applicants removed any genes where a majority GWAS effect direction could not be computed (i.e., an equal number of protective and risk effects), genes where eQTL risk variants shared a median slope that matched the median slope of eQTL protective variants (e.g. positive slope in both cases), and genes where the slope of any eQTL risk variant matches the slope of any eQTL protective variant. Outlier variants are then compared on the slope and GWAS effect direction of the consensus eQTL results (e.g., for a given gene with positive median slope and GWAS risk effect, Applicants assessed if the outlier variant was an over-expression outlier variant and its comparable relationship to GWAS risk).

Inferring UKBB non-British white validation cohort

Using the self-identified non-British white labels that were reported in the UKBB metadata, a larger cohort of predicted non-British white individuals was inferred. For all self-reported non-British white individuals, the mean and standard deviation of the first and second genotypic principal components were calculated. All individuals without a self-reported ethnic identity that were within +/−3 SD of the calculated mean PCI and PC2 values were inferred to be non-British white. All self-reported non-British white individuals that fell out of this range were also excluded. This final cohort consisted of 23,790 self-reported non-British individuals, and 97,154 inferred non-British white individuals. Applicants found that the PRS distribution of this non-British white cohort did not differ significantly from a normal distribution (Shapiro-Wilk normality test; P=0.2774), suggesting that the PRS as calculated on the British white cohort generalizes well to this cohort. Quantifying effect on phenotypes associated with IOGC score.

Using the list of GTEx outlier variants linked to genes, Applicants retained the genes in which >=1 outlier variant overlapped any PRS variants (within a +/−10 Kb window). In this way, Applicants focus only on genes previously linked to the phenotype (and therefore included in the PRS). The resulting set of outlier-and non-outlier variants in retained genes were written to a lookup file which was then input to the software plink (26) (—extract flag) to identify UKBB individuals in the validation cohort who are heterozygous or homozygous for each variant (i.e. alternate allele genotype 1 and 2, respectively). Applicants then used previously-released UKBB GWAS effect estimates to assign effect directions to each outlier variant (i.e., risk/protective). Given that the previously-released GWAS were calculated on UKBB individuals with white British genetic ancestry (see 31), the non-British white cohort validation cohort Applicants constructed for this study, as well as the TOPMed WHI cohort, was non-overlapping.

Applicants quantified the effect of outlier variant burden on phenotype by computing a score that summarizes, per individual, putative outlier gene burden. Applicants refer to this quantity as the independent outlier gene count (IOGC). To compute this score, for each individual Applicants link variants to effect size direction in UKBB, then collapse to gene-level to prevent double-counting. Per individual, Applicants convert the beta effect estimate per variant to integers using a sign function:

$$sgn ( {β}_{k} ) : = \{ \begin{matrix}- 1 & if {β}_{k} < 0 , \\ 0 & if {β}_{k} = 0 , \\ 1 & if {β}_{k} > 0 .\end{matrix}$$

where β is the UKBB GWAS beta coefficient for variant k. In practice, effect sizes of zero are not generally observed, so Applicants expect to see only values of −1 or 1. Following this step, Applicants take the distinct values per gene (i.e. remove duplicates); since the goal is to use outlier variants to tag outlier/dysregulated gene expression, this step prevents counting of putative outlier gene expression more than once. Therefore, if the vector of sgn(βₖ) is for variants linked to a given gene as s, then:

θ(s)={sᵢ}_{i∈{1, . . . n}}, where s =[sᵢ, . . . , Sₙ]

This is repeated across all genes (g) linked to >=1 outlier variant, and summed to yield the IOGC score for each individual (j):

$${IOGC}_{j} = {\sum }_{i = 1}^{g} {θ ( s )}_{i}$$

Linear regression was used for quantitative phenotypes, and logistic regression for binary phenotypes. In the regression models, Applicants adjusted for PRS, age, sex, first ten principal components of genetic ancestry, and genotyping array.

All statistical analyses were performed using R (version 3.6.0). Plots were generated using ggplot2 (version 3.3.0)³².

Resource availability

GTEx (v7) RNA-seq and WGS data is available from dbGaP (dbGaP Accession phs000424.v7.p2)

GTEx (v7) eQTL summary statistics were downloaded from the GTEx Portal available at the World

Wide Web gtexportal.org/home/datasets

GTEx v7 colocalization summary statistics were downloaded from LocusCompare the World Wide Web locuscompare.com

Data from the TOPMed Women's Health Initiative is available from dbGaP (dbGaP Accession phs000200.v12.p3)

UK Biobank (UKBB) data was obtained under application number 24983 (PI: Dr. Manuel Rivas) UKBB Phase 1 GWAS summary statistics were downloaded from the Neale Lab server available at the World Wide Web the World Wide Web nealelab.is/uk-biobank.

Polygenic risk scores (PRS) for body mass index and type-2 diabetes were downloaded from the Cardiovascular Disease Knowledge Portal available at kp4cd.org/dataset_downloads/mi.

Gene annotation data was obtained from GENCODE (version 19) available at the World Wide Web gencodegenes.org/human/release_19.html.

Allele frequency data was obtained from gnomAD (version r2.0.2) available at console.cloud.google.com/storage/browser/gnomad-public/release/2.0.2/hg19 coordinates were converted to hg38 using the chain file available at hgdownload.soe.ucsc.edu/goldenPath/hg19/liftOver/. Custom scripts to conduct all analyses not performed using existing software can be found at github.com/csmail/outlier_prs.

### Example 3

The analysis conducted in Examples 1 and 2 was conducted for breast cancer. The IOGC applied to breast cancer led to improved prediction of disease susceptibility in extremes of PRS-predicted disease risk. For example, individual subjects s in extreme of PRS “low-risk” (mean incidence of breast cancer=1.16%) but with a high IOGC score have a 5.26% incidence of breast cancer (a 4.5-fold increase in PRS-predicted disease risk). Similarly, for extreme PRS “high-risk” individual subjects (mean incidence of breast cancer=9%) and with high IOGC score, the incidence of breast cancer is 12.5% (a 1.38-fold increase in disease risk).

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### REFERENCES

1. Khera, A. V. et al. Polygenic Prediction of Weight and Obesity Trajectories from Birth to Adulthood. Cell 177, 587-596.e9 (2019).
2. Martin, A. R., Daly, M. J., Robinson, E. B., Hyman, S. E. & Neale, B. M. Predicting Polygenic Risk of Psychiatric Disorders. Biol. Psychiatry 86, 97-109 (2019).
3. Elliott, J. et al. Predictive Accuracy of a Polygenic Risk Score-Enhanced Prediction Model vs a Clinical Risk Score for Coronary Artery Disease. JAMA 323, 636-645 (2020).
4. Khera, A. V. et al. Genome-wide polygenic scores for common diseases identify individuals with risk equivalent to monogenic mutations. Nat. Genet. 50, 1219-1224 (2018).
5. Zhang, Y. D. et al. Assessment of polygenic architecture and risk prediction based on common variants across fourteen cancers.Nat. Commun. 11, 3353 (2020).
6. Aguilera, F. R.-M. et al. An integrated polygenic and clinical risk tool enhances coronary artery disease prediction. doi: 10.1101/2020.06.01.20119297.
7. Yang, J. et al. Genetic variance estimation with imputed variants finds negligible missing heritability for human height and body mass index. Nat. Genet. 47, 1114-1120 (2015).
8. Mancuso, N. et al. The contribution of rare variation to prostate cancer heritability. Nat. Genet. 48, 30-35 (2016).
9. Li, X. et al. The impact of rare variation on gene expression across tissues. Nature 550, 239-243 (2017).
10. Zhao, J. et al. A Burden of Rare Variants Associated with Extremes of Gene Expression in Human Peripheral Blood. Am. J. Hum. Genet. 98, 299-309 (2016).
11. Li, X. et al. Transcriptome sequencing of a large human family identifies the impact of rare noncoding variants. Am. J. Hum. Genet. 95, 245-256 (2014).
12. Zeng, Y. et al. Aberrant gene expression in humans. PLoS Genet. 11, e1004942 (2015).
13. Ferraro, N. M. et al. Diverse transcriptomic signatures across human tissues identify functional rare genetic variation. bioRxiv 786053 (2019) doi: 10.1101/786053.
14. Bonder, M. J., Smail, C., Gloudemans, M. J. & Frésard, L. Systematic assessment of regulatory effects of human disease variants in pluripotent cells. bioRxiv (2019).
15. Bycroft, C. et al. The UK Biobank resource with deep phenotyping and genomic data. Nature 562, 203-209 (2018).
16. Hurt, R. T., Kulisek, C., Buchanan, L. A. & McClave, S. A. The obesity epidemic: challenges, health initiatives, and implications for gastroenterologists. Gastroenterol. Hepatol. 6, 780 (2010).
17. Liu, B., Gloudemans, M. J., Rao, A. S., Ingelsson, E. & Montgomery, S. B. Abundant associations with gene expression complicate GWAS follow-up. Nat. Genet. 51, 768-769 (2019).
18. Hernandez, R. D. et al. Ultrarare variants drive substantial cis heritability of human gene expression. Nature Genetics vol. 51 1349-1355 (2019).
19. Eyre-Walker, A. Genetic architecture of a complex trait and its implications for fitness and genome-wide association studies. Proceedings of the National Academy of Sciences vol. 107 1752-1756 (2010).
20. Zeng, J. et al. Signatures of negative selection in the genetic architecture of human complex traits. Nat. Genet. 50, 746-753 (2018).
21. Quinlan, A. R. & Hall, I. M. BEDTools: a flexible suite of utilities for comparing genomic features.Bioinformatics 26, 841-842 (2010).
22. Pedersen, B. S., Layer, R. M. & Quinlan, A. R. Vcfanno: fast, flexible annotation of genetic variants. Genome Biol. 17, 118 (2016).
23. Karczewski, K. J. et al. The mutational constraint spectrum quantified from variation in 141,456 humans. Nature 581, 434-443 (2020).
24. Rentzsch, P., Witten, D., Cooper, G. M., Shendure, J. & Kircher, M. CADD: predicting the deleteriousness of variants throughout the human genome. Nucleic Acids Res. 47, D886-D894 (2019).
25. Stegle, O., Parts, L., Piipari, M., Winn, J. & Durbin, R. Using probabilistic estimation of expression residuals (PEER) to obtain increased power and interpretability of gene expression analyses. Nat. Protoc. 7, 500-507 (2012).
26. Purcell, S. et al. PLINK: a tool set for whole-genome association and population-based linkage analyses. Am. J. Hum. Genet. 81, 559-575 (2007).
27. Zhao, H. et al. CrossMap: a versatile tool for coordinate conversion between genome assemblies. Bioinformatics 30, 1006-1007 (2014).
28. Abraham, G., Qiu, Y. & Inouye, M. FlashPCA2: principal component analysis of Biobank-scale genotype datasets. Bioinformatics 33, 2776-2778 (2017).
29. Klarin, D. et al. Genetics of blood lipids among\ 300,000 multi-ethnic participants of the Million Veteran Program.Nat. Genet. 50, 1514-1523 (2018).
30. Pang, H. et al. Harmonizing Genetic Ancestry and Self-identified Race/Ethnicity in Genome-wide Association Studies. Am. J. Hum. Genet. 105, 763-772 (2019).
31. Churchhouse, C. Neale lab. (2017).
32. Wickham, H. ggplot2: Elegant Graphics for Data Analysis. (Springer, 2016).

What is claimed is:

1. A method of treating obesity in an individual subject, the method comprising administering to the individual subject a gene therapy to modify the genomic DNA in the individual subject corresponding to a genetic variant, wherein the genetic variant is 1:179969180:A:G, 1:201839818:G:A, 2:157365073:G:T, 4:8235241:G:A, 5:141309152:C:T, 6:5701905:C:T, 9:135493831:G:C, 16:89960355:G:T, 17:4864345:C:T, or 21:27244607:G:A.

2. The method of claim 1, wherein the gene therapy involves a modification of the genomic DNA by CRISPR, zinc finger nucleases, and/or meganuceases.

3. The method of claim 1, wherein the gene therapy involves a modification of the genomic DNA by a nucleic acid vector.

4. The method of claim 3, wherein the nucleic acid vector comprises a viral vector.

5. The method of claim 4, wherein the viral vector comprises at least one of retroviruses, adenoviruses, adeno-associated viruses, and lentiviruses.
